(19) United States
(12) Patent Application Publication
(10) Pub. No.: US 2005/0255123 A1
(43) Pub. Date: Nov. 17, 2005

(54) Chimeric ebola virus envelopes and uses therefor

(75) Inventors: James M. Wilson, Gladwyne, PA (US); Gary Kobinger, Philadelphia, PA (US); Maria Fe C. Medina, Hamilton (CA)

Correspondence Address: HOWSON AND HOWSON;ONE SPRING HOUSE CORPORATION CENTER, BOX 457, 321 NORRISTOWN ROAD, SPRING HOUSE, PA, 19477, US

(73) Assignee: The Trustees of the University of Pennsylvania, Philadephia, PA (US)

(21) Appl. No.: 10/510,947

(22) PCT Filed: Apr. 28, 2003
(86) PCT No.: PCT/US03/11494

§ 371 Date: Jan. 11, 2005

Related U.S. Application Data
(60) Provisional application No. 60/376,480, filed on Apr. 30, 2002.
(60) Provisional application No. 60/385,704, filed on Jun. 4, 2002.
(60) Provisional application No. 60/427,752, filed on Nov. 20, 2002.

(51) Int. Cl. C07K 14/01; C12Q 1/70; C07H 21/04; A61K 39/12; C12N 7/00

(52) U.S. Cl. 424/186.1; 435/5; 435/69.3; 435/235.1; 435/325; 530/350; 536/23.72

(57) ABSTRACT

Chimeric ebola envelope proteins and uses therefore are described. The chimeric envelope proteins are useful for packaging viral vectors and targeting these vectors in vivo, to lung cells following intratracheal delivery or for delivery of molecules, ex vivo, to macrophages and dendritic cells. In another aspect, also provided herein are immunogenic compositions which contain ebola envelope proteins and uses thereof.

### BACKGROUND OF THE INVENTION

The invention relates generally to recombinant viruses useful for delivery of transgenes and to antigens useful for generating an immune response to ebola virus.

What is needed is a safe vector useful for delivery of a therapeutic gene to a selected target cell. Further, what is desirable is a vector which can readily transduce the target cell using procedures in which invasiveness is minimized. Also desired are antigens useful in inducing an immune response to against ebola virus.

### SUMMARY OF THE INVENTION

The present invention provides chimeric ebola envelope proteins.

In one embodiment, the chimeric ebola envelope proteins are used to generate a chimeric ebola-pseudotyped virus which delivers a selected molecule to a target cell. In another embodiment, the virus of the invention contains lentiviral or other sources of viral sequences packaged into the chimeric ebola envelope protein of the invention.

Advantageously, the recombinant virus of the invention containing the chimeric ebola envelope proteins of the invention minimizes the safety concerns that the packaged virus will form replication competent virus. Further, in certain embodiments, the recombinant virus of the invention is particularly well adapted to delivery to mammalian lung cells, as the transfer virus infects from the apical side, permitting delivery via intracheal administration.

In another aspect, the chimeric ebola envelope proteins of the invention are delivered to host cells in a manner that induces an immune response to ebola. In one embodiment, immunogenic compositions are prepared which contain vectors carrying the sequences encoding the chimeric ebola envelope proteins under the control of regulatory sequences which direct expression thereof so that, upon expression in a host cell, an immune response to ebola virus is induced. In another aspect, an immunogenic composition of the invention contains one or more of the ebola virus envelope proteins of the invention, wild-type ebola envelope proteins, and/or mixtures thereof in a suitable immunogenic carrier.

These and other aspects of the invention will be readily apparent from the following detailed description of the invention.

### BRIEF DESCRIPTION OF THE DRAWINGS

FIGS. 1A through 1Q are schematic diagrams of chimeric filovirus envelope proteins of the invention based on the ebola Zaire (EboZ) strain glycoprotein. Hybrid envelopes were developed by replacing EboZ transmembrane (Tm) and cytoplasmic (Cyt) domains with corresponding regions from Vesicular Stomatitis Virus glycoprotein (VSV-G). FIG. 1A is a schematic diagram of the wild-type (wt) EboZ glycoprotein [SEQ ID NO:1]. FIG. 1B is a schematic diagram of chimeric eboZ glycoprotein V/TC which is deleted of the eboZ wt Tm and Cyt domains fused to an intact VSV tm domain (amino acids 463 to 511 of SEQ ID NO:3). FIG. 1C is a schematic diagram of chimeric eboZ glycoprotein −2aa, which is deleted of the wt Tm and Cyt domains fused to a shortened version of the VSV transmembrane domain (amino acids 465 to 511 of SEQ ID NO:3) and an intact VSV-G cytoplasmic domain. FIG. 1D is a schematic diagram of chimeric eboZ glycoprotein +2aa, in which the ebola envelope protein deleted of the wt tm and cyt domains is fused to a short sequence of VSV from the sequences 5′ to its Tm and Cyt, the Tm and Cyt domain (amino acids 461 to 511 of SEQ ID NO:3). FIG. 1E is a schematic diagram of chimeric eboZ glycoprotein +16aa, which is deleted of the wt Tm and Cyt domains and fused to a longer 5′ sequence from VSV-G, the VSV-G Tm domain and the Cyt domain of VSV-G (amino acids 447 to 511 of SEQ ID NO:3). FIG. 1F is a schematic diagram of a chimeric eboZ glycoprotein +23aa, in which the ebola envelope protein is deleted of the transmembrane and cytoplasmic domain and has been fused to a longer 5′ sequence from VSV-G, the VSV-G transmembrane domain and the cytoplasmic domain of VSV-G (amino acids 440 to 511 of SEQ ID NO:3). FIG. 1G is a schematic diagram of a chimeric eboZ glycoprotein deleted of the wt tm and cyt domains fused to a VSV Tm domain deleted (hatched) and increased in the 5′ region by +42aa to include a putative VSV-G budding domain. FIG. 1H is a schematic diagram of chimeric ebola V/C, in which the EboZ Tm domain was retained [truncated at amino acid 672, SEQ ID NO:1] and fused to the VSV-G Cyt domain without an intervening EboZ Cyt domain (amino acids 483 to 511 of SEQ ID NO:3). FIG. 1I a schematic diagram of chimeric ebola V/2C, in which the full-length ebola envelope protein is fused to the cytoplasmic domain of VSV-G (amino acids 483 to 511 of SEQ ID NO:3). FIG. 1J is a schematic diagram of chimeric EboZ envelope V/T, in which the ebola protein has a terminal truncation which removed the transmembrane and cytoplasmic domains (terminating at amino acid 650 of SEQ ID NO:1), and which is fused to the transmembrane domain of VSV-G (amino acids 463 to 482 of SEQ ID NO:3). FIG. 1K is a schematic diagram of chimeric EboZ envelope (Δint), in which a region immediately upstream of the EboZ Tm domain was deleted [truncated at amino acid 629, SEQ ID NO:1] and fused to sequences from VSV-G including the transmembrane and cytoplasmic domains. FIG. 1L is schematic diagram of chimeric EboZ envelope ΔImm, in which a region immediately upstream of the EboZ Tm domain was deleted [truncated at amino acid 563, SEQ ID NO:1] fused to sequences from VSV-G including the transmembrane and cytoplasmic domains. FIG. 1M is a chimeric EboZ envelope which contains the eboZ binding domain cloned in the VSV-G envelope (VE). FIG. 1N is a schematic diagram of chimeric EboZ envelope H/TC, which is composed of the ebola protein with a terminal truncation which removed the transmembrane and cytoplasmic domains (terminating at amino acid 650, SEQ ID NO:1), fused to the transmembrane domain of an HIV glycoprotein (amino acids 661 to 856, SEQ ID NO:8). FIG. 10 is schematic diagram of chimeric EboZ envelope M/C, which is composed of the ebola protein with a terminal truncation that removed the transmembrane and cytoplasmic domains of ebola, with the ebola truncate fused to a VSV-G transmembrane domain and an MLV-GP cytoplasmic domain (amino acids 634 to 649 of MLV-GP, SEQ ID NO:6). FIG. 1P is schematic diagram of chimeric EboZ envelope M/CR, which is composed of the M/C construct with an “R” peptide of MLV-GP (amino acids 650 to 665 of MLV-GP, SEQ ID NO:6). FIG. 1Q is schematic diagram of chimeric EboZ envelope L/TC, which is composed of the ebola protein with a terminal truncation that removed the wild-type transmembrane and cytoplasmic domain; fused to the terminal truncation is the transmembrane protein and cytoplasmic domain of lymphyocytic choriomeningitis virus (LCMV) glycoprotein (GP) (amino acids 439 to 498 of LCMV-GP, SEQ ID NO:9).

FIGS. 2A through 2O are schematic diagrams of chimeric filovirus envelope proteins of the invention based on the ebola Zaire strain glycoprotein. FIG. 2A is the wild-type EboZ glycoprotein. FIG. 2B is an EboZ deletion chimera created by deleting a region in the carboxy-terminal (EboΔCyt) region of the envelope. FIG. 2C is an EboZ deletion chimera created by deleting a region in the amino-terminal (EboΔN) region of the envelope. FIG. 2D is a chimeric ebola envelope created by removing 130 amino acids from the variable, mucin-rich region located downstream of the EboZ binding domain (NTLD1). FIG. 2E is a chimeric ebola envelope created by removing 135 amino acids from the variable, mucin-rich region located downstream of the EboZ binding domain (NTLD2). FIG. 2F is a chimeric ebola envelope created by removing 121 amino acids from the variable, mucin-rich region located downstream of the EboZ binding domain (NTLD3). FIG. 2G is a chimeric ebola envelope created by removing 185 amino acids from the variable, mucin-rich region located downstream of the EboZ binding domain (NTLD4). FIG. 2H is a chimeric ebola envelope created by removing 209 amino acids from the variable, mucin-rich region located downstream of the EboZ binding domain (NTLD5). FIG. 2I is a chimeric ebola envelope created by removing 217 amino acids from the variable, mucin-rich region located downstream of the EboZ binding domain (NTLD6). FIG. 2J is a chimeric ebola envelope created by removing 229 amino acids from the variable, mucin-rich region located downstream of the EboZ binding domain (NTLD7). FIG. 2K is a chimeric ebola envelope created by removing 238 amino acids from the variable, mucin-rich region located downstream of the EboZ binding domain (NTLD8). FIG. 2L is a chimeric ebola envelope created by removing 256 amino acids from the variable, mucin-rich region located downstream of the EboZ binding domain (NTLD9). FIG. 2M is a chimeric ebola envelope created by removing 270 amino acids from the variable, mucin-rich region located downstream of the EboZ binding domain (NTLD10). FIG. 2N is a schematic drawing of an internally deleted LCMV envelope chimera (ΔL1) developed in a similar manner. FIG. 2O is a schematic drawing of an internally deleted LCMV envelope chimera (ΔL2) developed in a similar manner.

FIGS. 3A through 3J are schematic diagrams of chimeric envelope proteins of the invention based on the ebola soluble glycoprotein. FIG. 3A is the wild-type EboZ envelope glycoprotein. FIG. 3B is the EboZ soluble glycoprotein. FIG. 3C is the EboZ glycoprotein with an internal deletion at amino acids 367496 [SEQ ID NO:1] (EboΔ1). FIG. 3D is the EboZ glycoprotein with an internal deletion at amino acids 367-501 [SEQ ID NO:1] (EboΔ2). FIG. 3E is the EboZ glycoprotein with an internal deletion at amino acids 367-491 [SEQ ID NO:1] (EboΔ3). FIG. 3F is the EboZ glycoprotein with an internal deletion at amino acids 312-496 [SEQ ID NO:1] (EboΔ4). FIG. 3G is the EboZ soluble glycoprotein with a terminal deletion at amino acid 220 [SEQ ID NO:2] (EboΔ5S). FIG. 3H is the EboZ soluble glycoprotein with a terminal deletion at amino acid 361 [SEQ ID NO:2] (EboΔ6). FIG. 3I is the EboZ soluble glycoprotein with a terminal deletion at amino acid 628 [SEQ ID NO:2] (EboΔ7S). FIG. 3J is the EboZ soluble glycoprotein with an internal deletion at amino acids 312-496 [SEQ ID NO:2] (EboΔ8S).

### DETAILED DESCRIPTION OF THE INVENTION

The present invention provides chimeric ebola virus envelope proteins. These proteins are useful in a variety of contexts, including as immunogens for generating an immune response to ebola when expressed in vivo.

In one aspect, the invention provides chimeric ebola virus envelope proteins that are useful for inducing an immune response against ebola. Such proteins can be expressed in vivo, e.g., following delivery via a viral or other suitable vector. Alternatively, the chimeric ebola virus envelope proteins can be delivered in protein form via a suitable carrier.

Advantageously, the invention includes chimeric ebola proteins that are as immunogenic as the wild-type (wt) envelope. The invention includes ebola chimera which are advantageous as compared to the wt envelope due to its lower cellular toxicity.

The invention further provides a recombinant virus containing a viral minigene in a chimeric ebola virus envelope protein, which is able to efficiently transduce intact airway epithelia ex vivo and more importantly in vivo. Thus, the viruses of the invention with the chimeric ebola envelope proteins are particularly well suited for delivery of molecules to airway cells, e.g., for treatment of cystic fibrosis. However, the constructs described herein are useful for targeting viral vectors to desired host cells, including lung cells, dendritic cells, and macrophages, among others.

Other advantages and uses of the proteins and viruses of the invention are described below and will readily apparent to those of skill in the art.

I. Chimeric Ebola Proteins

As used herein the term “chimeric ebola protein” includes proteins that contain, at a minimum, a functional ebola envelope protein binding domain. In one embodiment, the ebola envelope protein contains an intact, wild-type binding domain. However, the wild-type binding domain may be altered, so long as it provides a functional ebola binding domain. By “functional” is meant that that the ability of the envelope of the chimeric ebola protein of the invention retains the ability to bind to the target of the wild-type ebola binding domain. These chimeric ebola proteins include ebola envelope proteins with internal deletions, N-terminal deletions, COOH-terminal deletions, and combinations of such deletions and/or ebola envelope proteins with, or without, such deletions which are fused to a partner derived from a protein from other viral source, or a non-viral source. These chimeric ebola proteins can further contain other modifications to the protein sequences of the ebola protein, or the sequences expressing such proteins, e.g., to improve expression, yield, purification, or for other reasons. The chimeric ebola proteins of the invention are not limited by the method by which they are produced, which may be any suitable method including, e.g., recombinant means, chemical means, synthetic means, or by other suitable methods known to those of skill in the art. See, e.g., Sambrook et al, Molecular Cloning: A Laboratory Manual, Cold Spring Harbor Laboratories, Cold Spring Harbor, N.Y.

Thus, the invention provides chimeric filovirus envelope proteins. Suitably, the filovirus which provides the sequences encoding the envelope protein or a polypeptide or peptide thereof (e.g., the binding domain) is an Ebola virus, selected from any suitable serotype, e.g., Zaire, Sudan, Reston, or an artificial serotype. Most preferably, the envelope protein is obtained from the ebola glycoprotein. In filoviruses, the glycoprotein gene is the fourth gene (of seven) from the 3′ end of the negative strand RNA genome. Thus, each of the filoviruses contains a type of glycoprotein organization in which a secreted, non-structural protein is expressed in preference to the structural glycoprotein.

For purposes of convenience, the amino acid sequences of the Ebola Zaire strain glycoprotein used in the examples are reproduced in SEQ ID NO: 1; and the amino acid sequences of the Ebola Zaire strain S protein used in the examples herein are reproduced in SEQ ID NO: 2. References to the residue number of the ebola glycoproteins correspond to the numbering provided in these sequences. The invention is not limited to these amino acid sequences.

In the Ebola Zaire (eboZ) strain, the secreted glycoprotein is 50 to 70 kD, and the type 1 transmembrane form encodes a 120- to 150-kD glycoprotein that is incorporated into the virion. The first 295 amino acids of both proteins are identical in the Zaire strain, but the secreted glycoprotein (sGP) contains an additional 60 amino acids and the transmembrane glycoprotein (GP) contains another 381 COOH-terminal amino acid residues [A. Sanchez et al, J. Virol., 72(8):6442-6447 (1998)]. As these two glycoproteins are known to target different cell types, either may be selected, depending upon the target cell. However, as sGP binds to neutrophils, it may not be as desirable as GP, which binds to endothelial cells.

The wild-type eboZ envelope protein contains a signal peptide located at about amino acids 1 to 31; a binding domain located at amino acids 180 to 350; a transmembrane domain located at amino acids 650 to 673; and a cytoplasmic domain located at amino acids 673 to 676, SEQ ID NO:1. Similar structural glycoproteins may be readily obtained from the other Ebola viral strains, or from Marburg virus glycoprotein, which has been described in comparison to the Ebola virus genome [A. Sanchez et al, Virus. Res., 29(3):215-240 (September 1993)].

The sequences encoding the envelope protein may be obtained by any suitable means, including application of genetic engineering techniques to a viral source, chemical synthesis techniques, recombinant production, or combinations thereof. Suitable sources of the desired viral sequences are well known in the art, and include a variety of academic, non-profit, commercial sources, and from electronic databases. The method by which the sequences are obtained is not a limitation of the present invention. The sequences of the ebola glycoprotein are published and are available from a variety of sources, including GenBank and the on-line database at the National Institutes for Health, PubMed nucleotides and proteins. Based upon the information provided herein and what is known to those of skill in the art, one can readily substitute other ebola strain envelope proteins or fragments thereof in chimeric envelope proteins as described herein.

A. Chimeric Envelopes with an Internal Deletion

In one embodiment, a chimeric ebola envelope protein of the invention contains one or more internal deletions within the wild-type ebola region between the signal peptide and the binding domain. Such internal deletions can be in the range of about 1, 5, 10, 15, 20, 50, 75 or more amino acids to about 80, 120, 130, 140, or 150 amino acids in length. The precise length of a selected deletion may be readily determined by one of skill in the art, in view of the information provided herein. Additionally, or alternatively, such an internal deletion(s) can include a truncation within the signal peptide and/or the binding domain, with the proviso that a functional binding domain is retained. Suitably, a truncation in the signal peptide may remove all or a portion of the carboxy terminus of the signal peptide and can be involve removal of from about 1, 5, or 10 to 15, 20, 25 or 30 amino acids. Typically, a truncation in the binding domain is minimal (e.g., from about 1 to about 10 amino acids in length), so that the function of the binding domain is retained. Additionally, or alternatively, such an internal deletion(s) can include removal of all or a portion of the ebola transmembrane domain and a portion of the cytoplasmic domain.

In one embodiment, the invention provides chimera containing internal deletions of the EboZ envelope made by deleting the region between the signal sequence and the binding domain (EboΔN). In one series of envelopes, deletions are performed on the highly variable, mucin-rich, region located in GP₁. As this region also contains sequences which contribute to the toxicity of the EboZ envelope in vitro, these envelopes were referred to as the NTDL (non-toxic, deletion, lung) chimera.

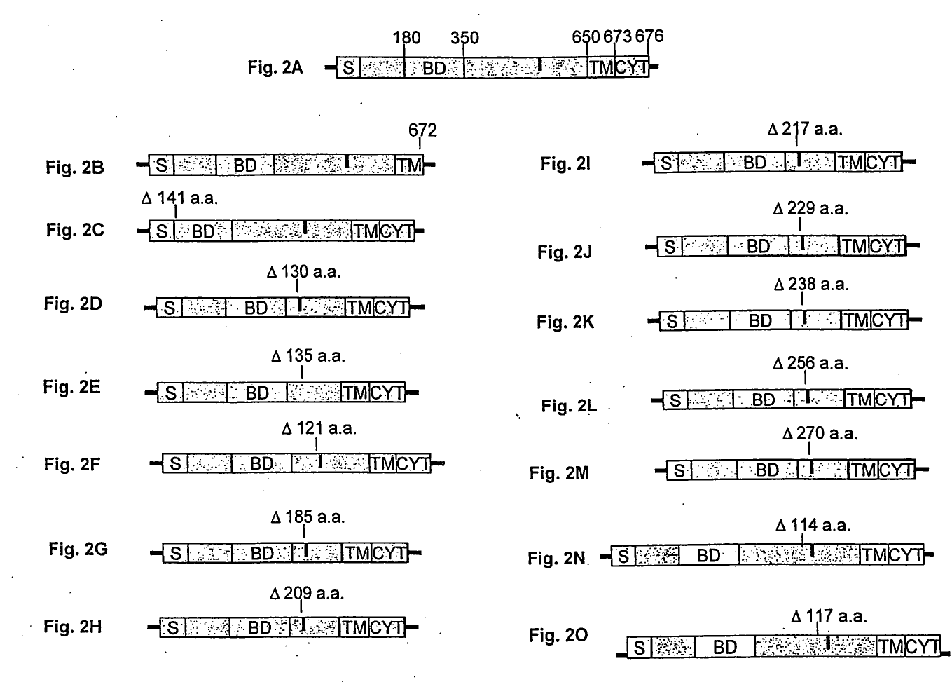

Examples of other internally deleted chimera include, without limitation, NTDL1, that refers to the ebola Z envelope protein [SEQ ID NO:1] with a deletion of amino acids 367 to 496 (i.e., amino acids 1 to 366 fused to 497 to 676); NTLD2, that refers to the ebola envelope protein with a deletion of amino acids 367 to 501 (i.e., amino acids 1 to 366 fused to 502 to 676); NTLD3, that refers to the ebola envelope protein with a deletion of amino acids 371 to 491 (i.e., amino acids 1 to 370 fused to 492 to 676); NTLD4, that refers to the ebola envelope protein with a deletion of amino acids 312 to 496 (i.e., amino acids 1 to 311 fused to 497 to 676). The furin recognition site (RRTRR, aa 497-501 of SEQ ID NO:1) was retained in NTDL1, NTDL3 and NTDL4 to allow post-translational cleavage of these envelopes. In NTDL2, the furin recognition site was also removed, preventing this envelope from being processed into GP₁ and GP₂. See, FIGS. 2A-2G.

Still other examples of internally deleted chimera include, without limitation, NTLD5, that refers to the ebola envelope protein [with reference to SEQ ID NO:1] with a deletion of amino acids 288 to 496 (i.e., amino acids 1 to 287 fused to 497 to 676); NTDL6, that refers to the ebola envelope protein with a deletion of amino acids 280 to 496 (i.e., amino acids 1 to 279 fused to 497 to 676); NTDL7, that refers to the ebola envelope protein with a 229 aa deletion at amino acids 268-496 (i.e., amino acids 1 to 267 fused to 497 to 676); NTDL8, that refers to the ebola envelope protein with a 238 aa deletion at amino acids 259-496 (i.e., amino acids 1 to 258 fused to 497 to 676); NTDL9, that refers to the ebola envelope protein with a deletion at residues 241 to 496; NTDL10, that refers to the ebola envelope protein with a deletion of amino acids 227 to 496 (i.e., amino acids 1 to 232 fused to 497 to 676); NTDL11, that refers to the ebola envelope protein with a deletion at residues 233-496; and NTDL13 refers to the ebola envelope protein with a deletion of amino acids 232-496 (i.e., amino acids 1 to 231 fused to 497 to 676). In yet another construct, termed ΔN, the ebola envelope protein has been deleted of amino acids 32 to 172 of the cytoplasmic domain so that it contains amino acids 1 to 31 fused to 172 to 676 of the EboZ env protein. See, FIGS. 2A-O.

Other suitable internal deletions will be readily apparent to one of skill in the art.

B. Chimeric Ebola Proteins with an Amino Terminal Deletion and/or Carboxy Terminal Truncations

In another embodiment, a chimeric ebola envelope protein of the invention can have an amino (NH₂—) terminal deletion and/or a carboxy (COOH—) terminal truncation in the ebola envelope protein. Amino terminal deletions can involve truncation or removal of the signal peptide. Such deletions can involve removal of from about 1, 5, or 10 amino acids to about 15, 20, 25 or 30 amino acids. In other embodiment, carboxy terminal truncations involve partial or complete removal of the transmembrane domain (about 1, 5, 10, 15 to about 20 to 23 amino acids), partial or complete removal of the cytoplasmic domain (about 1 to 3 amino acids), or combinations thereof. Additional amino deletions and/or carboxy truncations can be made, with the proviso that a functional ebola envelope binding domain is retained.

Examples of other suitable chimera include, with reference to SEQ ID NO:2, EboΔ5S which is truncated at amino acid 220 (removing amino acid 221 to 664 of the wild-type glycoprotein) and is secretable; EboΔ6S which is truncated at amino acid 361 (removing amino acid 362 to 664 of the wild-type glycoprotein) and is secretable; EboΔ7S which is truncated at amino acid 628 (removing amino acid 629 to 664 of the wild-type glycoprotein) and is secretable; and EboΔ8S which has an internal deletion at amino acids 312 to 496, so that amino acids 1 to 311 are fused to amino acids 497 to 664 of the wild-type glycoprotein, providing a secretable chimeric. These chimera are prepared using conventional techniques. Other chimera may be developed utilizing the secreted (S) protein.

Examples of suitable amino terminal deleted- or carboxy-terminal truncations are illustrated herein. For example, in the Δcyt construct, the ebola envelope protein has been deleted of the cytoplasmic domain so that it contains amino acids 1 to 672 of the EboZ env protein [SEQ ID NO:1].

In addition, a chimeric ebola protein of the invention can have an internal deletion and a carboxy terminal truncation, an internal deletion and an amino terminal deletion, or other combinations of deletions and/or truncations.

C. Chimeric Envelope Fusion Proteins

Optionally, a chimeric ebola envelope of the invention can be used to construct other chimera which are composed of the deleted and/or truncated ebola envelope fused to another proteins, and particularly viral envelope proteins.

Examples of suitable viral envelope proteins include vesicular stomatatis virus glycoprotein (VSV-G) [W. R. Beyer et al, J. Virol., 76(3):1488-1495 (2002); PubMed Accession No. CAC47944, encoded by AJ318514.1; protein sequence reproduced in SEQ ID NO:3; coding sequences for transmembrane and cytoplasmic domains reproduced in SEQ ID NO:4; coding sequences for 42 amino acids upstream of VSV-G transmembrane domain reproduced in SEQ ID NO:5; murine leukemia virus (MLV) [GenBank Accession No. AA061196; mature peptide coded by AF411814.1; envelope protein reproduced in SEQ ID NO:6 [propeptide gPr81, aa 1-654; signal peptide aa 1-30; mature env surface peptide, gp70, aa31-458; propeptide, aa 459-654; mature p12E protein, aa 459-639; mature R peptide, aa 640-654]; coding sequences for cytoplasmic and R domains reproduced in SEQ ID NO: 7; human immunodeficiency virus (HIV) 1 [GenBank Accession no. NP_{—}057856; mature peptide coded by NC_{—}001802.1; reproduced in SEQ ID NO: 8 [gp160, aa 1-856; signal peptide, aa 1-28; mature gp120, aa 29-511; mature gp41, aa 512-856] and lymphyocytic choriomeningitis virus (LCMV) [P J Southern et al, Virol., 157(10):145-155 (1987); GenBank Accession No. P09991; envelope protein reproduced in SEQ ID NO: 9; coding sequences for transmembrane and cytoplasmic domain reproduced in SEQ ID NO: 10. Desirably, the cytoplasmic domains or transmembrane domains of these viral envelope proteins is used in a fusion protein of the invention. Optionally, other proteins, e.g., the R protein of MLV [aa 640-654 of SEQ ID NO:6], can be used in the fusion proteins of the invention Alternatively, other suitable fusion partners may be readily selected from other viral or non-viral sources. Optionally, linkers may be inserted between the sequences encoding the ebola envelope protein of the invention and the sequences encoding the fusion partner.

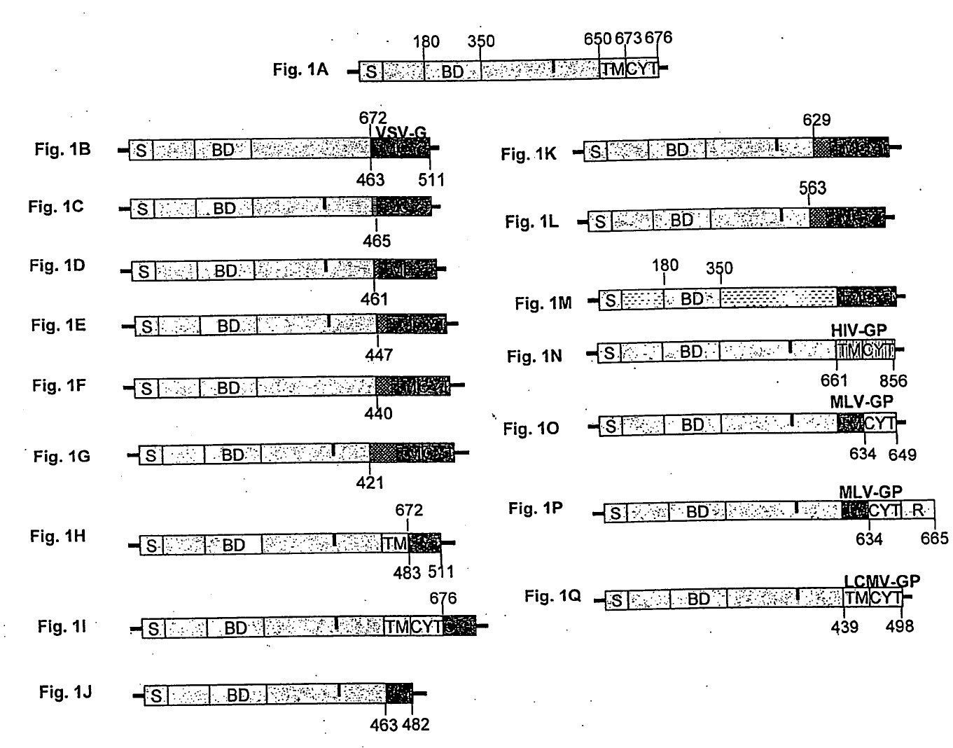

EboZ/VSV-G hybrid envelopes were constructed by fusing the amino terminal region of EboZ envelope containing the receptor-binding domain to the carboxy terminal region of VSV-G containing the TM and CYT domains. In these hybrids, the junction of the fused EboZ/VSV-G sequences is located in GP₂. Still other exemplary chimeric ebo envelopes lack a CYT domain. See, e.g., FIGS. 1A-1Q.

Optionally, the construct further contains a transmembrane domain which may of an origin heterologous to the ebola binding domain. For example, the transmembrane domain may be from a VSV-G transmembrane domain. Additionally or alternatively, the ebola envelope protein may further contain a cytoplasmic domain which may be heterologous to the ebola binding domain.

However, the invention is not limited to the specifically illustrated chimeric constructs. Suitable techniques for constructing such proteins are well known to those of skill in the art. See, generally, Sambrook et al, Molecular Cloning: A Laboratory Manual, Cold Spring Harbor Laboratories, Cold Spring Harbor, N.Y.

II. Immunization with Chimeric Ebola Proteins

In another aspect, the chimeric ebola envelope proteins of the invention and the sequences encoding them can be used to induce an immune response to ebola virus in a subject. Suitably, the proteins or coding sequences of the invention are delivered to the cells in a manner which presents them favorably for induction of an antibody response, a cellular immune response, or both. Any of the ebola envelope proteins described herein may be utilized in this aspect of the invention.

In one embodiment, an ebola envelope protein is delivered to the subject in protein form by a non-viral delivery vehicle. For example, liposomes, micelles, gels, multiple antigen complexes can be prepared utilizing the ebola envelope proteins described herein. A suitable immunogenic amount of the chimeric ebola envelope protein may be readily determined. For example, about 1 μg to 10 mg of protein may delivered via a suitable carrier. Optionally, the envelope protein of the invention can be conjugated with a polyethylene glycol conjugate according to published techniques. [See, e.g., U.S. Pat. No. 6,399,385 and references cited therein.]

In another embodiment, the sequences encoding a chimeric ebola envelope protein of the invention is delivered to the cell via a vector. As used herein, the term “vector” encompasses any suitable genetic element which delivers the sequences encoding the construct of the invention to a target cell, including, e.g., a plasmid, a transposon, a cosmid, an episome, a virus, or any other suitable molecule. Such vectors can be readily constructed using techniques known to those of skill in the art, utilizing vector elements known to those of skill in the art including, among others, the vector elements described in this application.

In one embodiment, a viral vector is utilized for delivery. Any suitable viral system can be used including, e.g., adenoviruses, poxviruses, and the like. For example, an adenoviral vector can be constructed using conventional techniques, which contains the sequences encoding a chimeric ebola envelope protein of the invention under the control of regulatory sequences that direct its expression in a cell. Suitably, such an adenoviral vector may be derived from any suitable human or non-human adenovirus, including, for example, any of human adenovirus serotypes 1 to 40, any of the chimpanzee adenovirus serotypes (e.g., Pan 9), or other non-human primate or non-human mammalian serotypes which will transduce the selected target cell. Suitable adenoviruses are readily available from the American Type Culture Collection, Manassas, Va. Alternatively, another viral or non-viral vector may be selected. Selection of an appropriate vector is not a limitation of the present invention. Appropriate amounts for vector-mediated delivery of the chimeric ebola envelope sequences can be readily determined by one of skill in the art, based on the information provided herein.

The vectors are administered in sufficient amounts to transduce the target cells and to provide a sufficient level of expression to provide an immune response without undue adverse or with medically acceptable physiological effects, which can be determined by those skilled in the medical arts. Conventional and pharmaceutically acceptable routes of administration include, but are not limited to, direct delivery to the target organ, inhalation, intraocular, intranasal, intravenous, intramuscular, intratracheal, subcutaneous, intradermal, rectal, oral and other parenteral routes of administration. Routes of administration may be combined, if desired, or adjusted depending upon the transgene or the condition. The route of administration primarily will depend on the nature of the condition being treated.

Dosages of the viral vector will depend primarily on factors such as the age, weight and health of the patient, and may thus vary among patients. For example, an effective adult human or veterinary dosage of the viral vector is generally in the range of from about 100 μL to about 100 mL of a carrier containing concentrations of from about 1×10⁶ to about 1×10¹⁵ particles, about 1×10¹¹ to 1×10¹³ particles, or about 1×10⁹ to 1×10¹² particles virus. Dosages will range depending upon the size of the animal and the route of administration. For example, a suitable human or veterinary dosage (for about an 80 kg animal) for intramuscular injection is in the range of about 1×10⁹ to about 5×10¹² particles per mL, for a single site. Optionally, multiple sites of administration may be delivered. In another example, a suitable human or veterinary dosage may be in the range of about 1×10¹¹ to about 1×10¹⁵ particles for an oral formulation. One of skill in the art may adjust these doses, depending the route of administration, and the therapeutic or vaccinal application for which the recombinant vector is employed. The levels of expression of the ebola protein, or for an immunogen, the level of circulating antibody, can be monitored to determine the frequency of dosage administration. Yet other methods for determining the timing of frequency of administration will be readily apparent to one of skill in the art.

The vaccinal and immunogenic compositions of the invention are formulated in a suitable delivery vehicle. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present invention.

Optionally, a composition of the invention may be formulated to contain other components, including, e.g. adjuvants, stabilizers, pH adjusters, preservatives and the like. Suitable exemplary adjuvants include, among others, liposomes, alum, monophosphoryl lipid A, immune-stimulating complexes (ISCOMS), LPS analogs including 3-O-deacylated monophosphoryl lipid A (Ribi Immunochem Research, Inc.; Hamilton, Mont.), mineral oil and water, aluminum hydroxide, Amphigen, Avirdine, L121/squalene, muramyl peptides, and saponins, such as Quil A, and any biologically active factor, such as cytokine, an interleukin, a chemokine, a ligands, and optimally combinations thereof. Certain of these biologically active factors can be expressed in vivo, e.g., via a plasmid or viral vector. For example, such an adjuvant can be administered with a priming DNA vaccine encoding an antigen to enhances the antigen-specific immune response compared with the immune response generated upon priming with a DNA vaccine encoding the antigen only. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin. Other suitable components for inclusion in an immunogenic composition of the invention are well known to those of skill in the vaccine art.

The levels of immunity of the selected gene can be monitored to determine the need, if any, for boosters. Following an assessment of antibody titers in the serum, optional booster immunizations may be desired.

In the examples below, experiments that evaluated humoral and cellular immune responses following vaccination of mice with adenoviral vector expressing different deletion chimera of the Ebola envelope glycoprotein of the invention are described. The inventors have found that the wild-type EboGP and an exemplary construct of the invention, the non-toxic deletion chimeric 4 (NTD4), induced substantial T and B cells responses. Similar frequencies of CD8+ T cells secreting INF-γ were observed in mice vaccinated with EboGP or NTD4. Serum from vaccinated mice with EboGP or NTD4 inhibited Ebola GP pseudotyped HIV vector mediated transduction with comparable efficiencies. The immune response induced by EboGP or NTD4 protected mice against lethal challenge performed with massive doses of a mouse-adapted strain of Ebola Zaire virus. Interestingly, a single administration of Ad-mediated EboGP or NTD4 fully protected mice against Ebola as early as 10 days post-vaccination. Thus, the present invention provides constructs useful in inducing an immune response, including a protective immune response, against ebola.

III. Recombinant Viruses

In one embodiment, the invention provides a recombinant virus composed of a viral vector carrying a heterologous molecule which is packaged in a heterologous envelope comprising a chimeric ebola virus envelope protein. Preferably, the envelope in which the viral minigene is packaged is suitably free of envelope protein from the source of the viral vector carrying the minigene. The chimeric ebola virus envelope proteins are used to target vectors derived from a viral source which is natively non-enveloped, including, without limitation, adenoviruses and adeno-associated viruses. For use in the present invention, the capsid proteins of such viral vectors can be provided with a lipid bilayer using published techniques, onto which the chimeric ebola envelope proteins of the invention can be engineered. Alternatively, the chimeric ebola envelope proteins of the invention are useful in providing a heterologous envelope to any vector derived from a viral source which natively contain has an envelope, e.g., retroviruses.

The heterologous molecule carried on the minigene for delivery to a host cell may be any desired substance including, without limitation, a polypeptide, protein, enzyme, carbohydrate, chemical moiety, or nucleic acid molecule which may include oligonucleotides, RNA, DNA, and/or RNA/DNA hybrids. In one embodiment, the heterologous molecule is a nucleic acid molecule which introduces specific genetic modifications into human chromosomes, e.g., for correction of mutated genes. In another desirable embodiment, the heterologous molecule comprises a transgene comprising a nucleic acid sequence encoding a desired protein, peptide, polypeptide, enzyme, or another product and regulatory sequences directing transcription and/or translation of the encoded product in a host cell, and which enable expression of the encoded product in the host cell. Suitable products and regulatory sequences are discussed in more detail below. However, the selection of the heterologous molecule carried on the minigene and delivered by the viruses of the invention is not a limitation of the present invention.

Methods of producing the viruses of the invention using packaging host cells are described below.

A. Viral Elements

Suitable viral elements for packaging into the chimeric envelope proteins of the invention may be readily determined by one of skill in the art. In one embodiment, the viral elements are selected from among lentiviral sources.

1. Lentiviral Vectors

In one embodiment, a lentiviral minigene is pseudotyped into a chimeric envelope protein of the invention. Suitable lentiviral minigenes are described in detail in WO 01/83730, which is incorporated by reference herein. See, also, WO 00/55335 (Sep. 21, 2000); WO 00/08131 (Feb. 17, 2000); WO 00/00600 (Jan. 6, 2000); WO 99/61589 (Dec. 2, 1999).

In selecting the lentiviral elements described herein for construction of a lentivirus minigene and pseudotyped lentivirus of the invention, one may readily select sequences from any suitable lentivirus and any suitable lentivirus serotype or strain. Suitable lentiviruses include, for example, human immunodeficiency virus (HIV), simian immunodeficiency virus (SIV), caprine arthritis and encephalitis virus, equine infectious anemia virus, bovine immunodeficiency virus, visna virus, and feline immunodeficiency virus (FIV). The examples provided herein illustrate the use of minigenes derived from HIV and FIV. However, other lentiviruses of human or non-human origin may also be particularly desirable. The sequences used in the constructs of the invention may be derived from academic, non-profit (e.g., the American Type Culture Collection, Manassas, Va.) or commercial sources of lentiviruses. Alternatively, the sequences may be produced recombinantly, using genetic engineering techniques, or synthesized using conventional techniques (e.g., G. Barony and R. B. Merrifield, THE PEPTIDES: ANALYSIS, SYNTHESIS & BIOLOGY, Academic Press, pp. 3-285 (1980)), with reference to published viral sequences, including sequences contained in publicly accessible electronic databases. In the following specification, it will be understood that a reference to lentiviral sequences involves any suitable means of obtaining the referenced sequences.

a. LTR Sequences

The lentiviral minigene contains a sufficient amount of lentiviral long terminal repeat (LTR) sequences to permit reverse transcription of the genome, to generate cDNA, and to permit expression of the RNA sequences present in the lentiviral minigene. Suitably, these sequences include both the 5′ LTR sequences, which are located at the extreme 5′ end of the minigene and the 3′ LTR sequences, which are located at the extreme 3′ end of the minigene. These LTR sequences may be intact LTRs native to a selected lentivirus or a cross-reactive lentivirus, or more desirably, may be modified LTRs.

Various modifications to lentivirus LTRs have been described. One particularly desirable modification is a self-inactivating LTR, such as that described in H. Miyoshi et al, J. Virol., 72:8150-8157 (October 1998) for HIV. In these HIV LTRs, the U3 region of the 5′ LTR is replaced with a strong heterologous promoter (e.g., CMV) and a deletion of 133 bp is made in the U3 region of the 3′ LTR. Thus, upon reverse transcription, the deletion of the 3′ LTR is transferred to the 5′ LTR, resulting in transcriptional inactivation of the LTR. The complete nucleotide sequence of HV is known. See, L. Ratner et al, Nature, 313(6000):277-284 (1985). Yet another suitable modification involves a complete deletion in the U3 region, so that the 5′ LTR contains only a strong heterologous promoter, the R region, and the U5 region; and the 3′ LTR contains only the R region, which includes a polyA. In yet another embodiment, both the U3 and U5 regions of the 5′ LTRs are deleted and the 3′ LTRs contain only the R region. These and other suitable modifications may be readily engineered by one of skill in the art, in HIV and/or in comparable regions of another selected lentivirus.

Optionally, the lentiviral minigene may contain a Ψ (psi) packaging signal sequence downstream of the 5′ lentivirus LTR sequences. Optionally, one or more splice donor sites may be located between the LTR sequences and immediately upstream of the Ψ sequence. According to the present invention, the Ψ sequences may be modified to remove the overlap with the gag sequences and to improve packaging. For example, a stop codon may be inserted upstream of the gag coding sequence. Other suitable modifications to the Ψ sequences may be engineered by one of skill in the art. Such modifications are not a limitation of the present invention.

In one suitable embodiment, the lentiviral minigene contains lentiviral Rev responsive element (RRE) sequences located downstream of the LTR and sequences. Suitably, the RRE sequences contain a minimum of about 275 to about 300 nt of the native lentiviral RRE sequences, and more preferably, at least about 400 to about 450 nt of the RRE sequences. Optionally, the RRE sequences may be substituted by another suitable element which assists in expression of gag/pol and its transportation to the cell nucleus. For example, other suitable sequences may include the CT element of the Manson-Pfizer virus, or the woodchuck hepatitis virus post-regulatory element (WPRE). Alternatively, the sequences encoding gag and gag/pol may be altered such that nuclear localization is modified without altering the amino acid sequences of the gag and gag/pol polypeptides. Suitable methods will be readily apparent to one of skill in the art.

Optionally, the pseudotyped lentivirus may contain other lentiviral elements, such as are well known in the art, many of which are described below in connection with the lentiviral packaging sequences. However, notably, the lentivirus minigene lacks the ability to assemble lentiviral envelope protein. Such a lentivirus minigene may contain a portion of the envelope sequences corresponding to the RRE, but lack the other envelope sequences. However, more desirably, the lentivirus minigene lacks the sequences encoding any functional lentiviral envelope protein in order to substantially eliminate the possibility of a recombination event which results in replication competent virus.

Thus, the lentiviral minigene of the invention contains, at a minimum, lentivirus 5′ long terminal repeat (LTR) sequences, (optionally) a psi encapsidation sequence, a molecule for delivery to the host cells, and a functional portion of the lentivirus 3′ LTR sequences. Desirably, the minigene further contains RRE sequences or their functional equivalent.

2. Non-Lentiviral Viral Sources

The invention further provides viruses containing the chimeric envelope proteins of the invention in which are packaged vectors from non-lentiviral sources carrying the minigenes. Suitable sources of viral vectors include viruses which natively contain an envelope protein, including, without limitation, retroviruses.

Other sources include viruses which are natively non-enveloped, but which have been modified so as to be conducive to packaging into the envelope proteins of the invention. For example, it may be desirable to modify an adenovirus or adeno-associated virus capsid protein for packaging into a chimeric envelope protein of the invention. Suitable techniques include providing the capsid protein of such viral vectors with a lipid coating (e.g., a lipid membrane or bilayer) onto which the chimeric envelope protein adheres. These and other techniques for facilitating association of the capsid proteins with envelope proteins are available in textbooks and the literature.

3. Transgene

The selected viral vector packaged into chimeric envelope protein of the invention carries the molecule for delivery to the target cells and other necessary regulatory sequences and vector elements. Desirably, the molecule carried by the minigene is a transgene. The transgene a nucleic acid molecule comprising a nucleic acid sequence, heterologous to the lentiviral sequences, which encodes a protein, peptide, polypeptide, enzyme, or another product of interest and regulatory sequences directing transcription and/or translation of the encoded product in a host cell, and which enable expression of the encoded product in the host cell. The composition of the transgene depends upon the intended use for the minigene and the virus of the invention.

For example, one type of transgene comprises a reporter or marker sequence which, upon expression, produces a detectable signal. Such reporter or marker sequences include, without limitation, DNA sequences encoding β-lactamase, β-galactosidase (LacZ), alkaline phosphatase, thymidine kinase, green fluorescent protein (GFP), chloramphenicol acetyltransferase (CAT), luciferase, membrane bound proteins including, for example, CD2, CD4, CD8, and the influenza hemagglutinin protein, as well as others well known in the art. Advantageously, high affinity antibodies to such proteins exist or can be made routinely, as can fusion proteins comprising a membrane bound protein appropriately fused to an antigen tag domain from, among others, hemagglutinin or Myc. These sequences, when associated with regulatory elements which drive their expression, provide signals detectable by conventional means. Such conventional means include enzymatic, radiographic, calorimetric, fluorescence or other spectrographic assays, fluorescent activated cell sorting assay and immunological assays, including enzyme linked immunosorbent assay (ELISA), radioimmunoassay (RIA) and immunohistochemistry. For example, where the transgene comprises the LacZ gene, the presence of the virus containing the lentiviral minigene is detected by assays for beta-galactosidase activity. Similarly, where the transgene is luciferase, virus may be measured by light production in a luminometer.

However, desirably, the transgene contains a non-marker gene which can be delivered to a cell or an animal via the virus of the invention. The transgene may be selected from a wide variety of gene products useful in biology and medicine, such as proteins, antisense nucleic acids (e.g., RNAs), or catalytic RNAs. The viruses of the invention are useful for delivery of gene products and other molecules which induce an antibody and/or cell-mediated immune response, e.g., for vaccine purposes. Suitable gene products may be readily selected by one of skill in the art from among immunogenic proteins and polypeptides derived from viruses, as well as from prokaryotic and eukaryotic organisms, including unicellular and multicellular parasites. In another alternative, the recombinant viruses of the invention are useful for delivery of a molecule desirable for study.

In one particularly desirable embodiment, the viruses of the invention are useful for therapeutic purposes, including, without limitation, correcting or ameliorating gene deficiencies, wherein normal genes are expressed but at less than normal levels. The viruses may also be used to correct or ameliorate genetic defects wherein a functional gene product is not expressed. A preferred type of transgene contains a sequence encoding a desired therapeutic product for expression in a host cell. These therapeutic nucleic acid sequences typically encode products which, upon expression, are able to correct or complement an inherited or non-inherited genetic defect, or treat an epigenetic disorder or disease. Alternatively, where it is desirable to down-regulate protein expression, a dominant negative mutant or an antisense sequence may be delivered.

The invention includes methods of producing a virus which can be used to correct or ameliorate a gene defect caused by a multi-subunit protein. In certain situations, a different transgene may be used to encode each subunit of the protein. This is desirable when the size of the DNA encoding the protein subunit is large, e.g., for an immunoglobulin or the platelet-derived growth factor receptor. In order for the cell to produce the multi-subunit protein, a cell would be infected with viruses containing each of the different subunits. Alternatively, different subunits of a protein may be encoded by the same transgene. In this case, a single transgene would include the DNA encoding each of the subunits, with the DNA for each subunit separated by an internal ribosome entry site (IRES). This is desirable when the size of the DNA encoding each of the subunits is small, such that the total of the DNA encoding the subunits and the IRES is less than nine kilobases. Alternatively, other methods which do not require the use of an IRES may be used for co-expression of proteins. Such other methods may involve the use of a second internal promoter, an alternative splice signal, or a co- or post-translational proteolytic cleavage strategy, among others which are known to those of skill in the art.

The selection of the transgene sequence, or other molecule carried by a minigene, is not a limitation of this invention. Choice of a transgene sequence is within the skill of the artisan in accordance with the teachings of this application.

4. Regulatory Elements

Design of a transgene or another nucleic acid sequence that requires transcription, translation and/or expression to obtain the desired gene product in cells and hosts may include appropriate sequences that are operably linked to the coding sequences of interest to promote expression of the encoded product. “Operably linked” sequences include both expression control sequences that are contiguous with the nucleic acid sequences of interest and expression control sequences that act in trans or at a distance to control the nucleic acid sequences of interest.

Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. A great number of expression control sequences—native, constitutive, inducible and/or tissue-specific—are known in the art and may be utilized to drive expression of the gene, depending upon the type of expression desired. For eukaryotic cells, expression control sequences typically include a promoter, an enhancer, such as one derived from an immunoglobulin gene, SV40, cytomegalovirus, etc., and a polyadenylation sequence which may include splice donor and acceptor sites. For a lentiviral vector, the polyadenylation (polyA) sequence generally is inserted following the transgene sequences and before the 3′ lentivirus LTR sequence. In one embodiment, the minigene carrying the transgene or other molecule contains the polyA from the lentivirus providing the LTR sequences, e.g., HIV. However, another source of polyA may be readily selected for inclusion in the construct of the invention. In one embodiment, the bovine growth hormone polyA is selected. A minigene of the present invention may also contain an intron, desirably located between the promoter/enhancer sequence and the transgene. One possible intron sequence is also derived from SV-40, and is referred to as the SV-40 T intron sequence. Another element that may be used in the vector is an internal ribosome entry site (IRES). An IRES sequence is used to produce more than one polypeptide from a single gene transcript. An IRES sequence would be used to produce a protein that contains more than one polypeptide chain. Selection of these and other common vector elements are conventional and many such sequences are available (see, e.g., Sambrook et al, and references cited therein at, for example, pages 3.18-3.26 and 16.17-16.27 and Ausubel et al., CURRENT PROTOCOLS IN MOLECULAR BIOLOGY, John Wiley & Sons, New York, 1989).

In one embodiment, high-level constitutive expression will be desired. Examples of useful constitutive promoters include, without limitation, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the β-actin promoter, the phosphoglycerol kinase (PGK) promoter, and the EF1 promoter (Invitrogen). Inducible promoters, regulated by exogenously supplied compounds, are also useful and include, the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al, Proc. Natl. Acad. Sci. USA, 93:3346-3351 (1996)), the tetracycline-repressible system (Gossen et al, Proc. Natl. Acad. Sci. USA, 89:5547-5551 (1992)), the tetracycline-inducible system (Gossen et al, Science, 268:1766-1769 (1995), see also Harvey et al, Curr. Opin. Chem. Biol., 2:512-518 (1998)), the RU486-inducible system (Wang et al, Nat. Biotech, 15:239-243 (1997) and Wang et al, Gene Ther., 4:432-441 (1997)) and the rapamycin-inducible system (Magari et al, J. Clin. Invest., 100:2865-2872 (1997)). Other types of inducible promoters which may be useful in this context are those which are regulated by a specific physiological state, e.g., temperature, acute phase, a particular differentiation state of the cell, or in replicating cells only.

In another embodiment, the native promoter for the transgene will be used. The native promoter may be preferred when it is desired that expression of the transgene mimic the native expression. The native promoter may be used when expression of the transgene must be regulated temporally or developmentally, in a tissue-specific manner, or in response to specific transcriptional stimuli. In a further embodiment, other native expression control elements, such as enhancer elements, polyadenylation sites or Kozak consensus sequences may also be used to mimic the native expression.

Another embodiment of the transgene includes a transgene operably linked to a tissue-specific promoter. For instance, if expression in skeletal muscle is desired, a promoter active in muscle should be used. These include the promoters from genes encoding skeletal β-actin, myosin light chain 2A, dystrophin, muscle creatine kinase, as well as synthetic muscle promoters with activities higher than naturally-occurring promoters (see Li et al., Nat. Biotech., 17:241-245 (1999)). Examples of promoters that are tissue-specific are known for liver (albumin, Miyatake et al. J. Virol., 71:5124-32 (1997); hepatitis B virus core promoter, Sandig et al., Gene Ther., 3:1002-9 (1996); alpha-fetoprotein (AFP), Arbuthnot et al., Hum. Gene Ther., 7:1503-14 (1996)), bone osteocalcin (Stein et al., Mol. Biol. Rep., 24:185-96 (1997)); bone sialoprotein (Chen et al., J. Bone Miner. Res., 11:654-64 (1996)), lymphocytes (CD2, Hansal et al., J. Immunol., 161:1063-8 (1998); immunoglobulin heavy chain; T cell receptor chain), neuronal such as neuron-specific enolase (NSE) promoter (Andersen et al. Cell. Mol. Neurobiol., 13:503-15 (1993)), neurofilament light-chain gene (Piccioli et al., Proc. Natl. Acad. Sci. USA, 88:5611-5 (1991)), and the neuron-specific vgf gene (Piccioli et al., Neuron, 15:373-84 (1995)), among others.

One of skill in the art can make a selection among these expression control sequences without departing from the scope of this invention. Suitable promoter/enhancer sequences may be selected by one of skill in the art using the guidance provided by this application. Such selection is a routine matter and is not a limitation of the molecule or construct. For instance, one or more expression control sequences may be operably linked to the coding sequence of interest, and inserted into the transgene, the minigene, and the virus of the invention. After following one of the methods for packaging the minigene taught in this specification, or as taught in the art, one may infect suitable cells in vitro or in vivo. The number of copies of the minigene in the cell may be monitored by Southern blotting or quantitative PCR. The level of RNA expression may be monitored by Northern blotting or quantitative RT-PCR. The level of expression may be monitored by Western blotting, immunohistochemistry, ELISA, RIA, or tests of the gene product's biological activity. Thus, one may easily assay whether a particular expression control sequence is suitable for a specific product encoded by the transgene, and choose the expression control sequence most appropriate. Alternatively, where the molecule for delivery does not require expression, e.g., a carbohydrate, polypeptide, peptide, etc., the expression control sequences need not form part of the lentiviral minigene or other molecule.

Suitably, a minigene of the invention is delivered to a host cell for packaging into a virus by any suitable means, e.g., by transfection of the “naked” DNA molecule comprising the minigene or by a vector which may contain other viral and regulatory elements described above, as well as any other elements commonly found on vectors. As defined above, a “vector” is any suitable vehicle which is capable of delivering the sequences or molecules carried thereon to a cell. Plasmids are particularly desirable for use in this aspect of the invention, although not required. The selected vector may be delivered by any suitable method, including transfection, electroporation, liposome delivery, membrane fusion techniques, high velocity DNA-coated pellets, viral infection and protoplast fusion.

According to the present invention, the minigene is packaged in a chimeric ebola envelope using the methods described herein to form the virus of the invention.

The inventors have found that deletion chimeric EboZ envelopes that, when used to pseudotype lentiviral vectors as used herein, increased titers up to 15-fold compared to the wild type EboZ envelope have been developed. These chimeric envelopes of the invention are incorporated within lentiviral vector particles with the same efficiency as the wild type EboZ envelope. Chimeric Ebo-pseudotyped lentiviral vectors with titers up to 1.5 logs higher compared to previous conditions have been produced. As an alternative to HIV-based vectors, lentiviral vector derived from feline immunodeficiency virus (FIV) that does not cause disease in humans, and has not been known to infect humans despite the extensive interaction of humans with cats have been used. FIV vectors were pseudotyped with either the wild type or the chimeric EboZ envelopes and injected intratracheally into C57Bl/6 mice. Results show that wild type, and more efficiently, chimeric EboZ envelopes were able to direct FIV vector transduction of airway epithelia from the apical side. Thus, chimeric Ebo envelopes of the invention can be used to generate higher titers of pseudotyped lentiviral vectors, retain the same tropism as the wild type Ebo envelope, allows higher efficiency of transduction of airway cells in vivo, while at the same time possesses minimal Ebo sequences than wild type Ebo envelope. Use of the chimeric envelope constructs of the invention for pseudotyping also provides advantages for large-scale production of these vectors.

IV. Production of Recombinant Virus

The invention further involves a method of producing a recombinant virus useful for delivering a selected molecule to a host cell.

In one embodiment, in vitro packaging techniques may be utilized, in which the envelope protein is produced in a host cell using the techniques described herein, extracted using conventional protein extraction techniques, and used to package the virus in vitro.

The nucleic acid molecule carrying the sequences encoding the envelope protein operably linked to its expression control sequences as described above, may be readily selected from among any suitable genetic element (i.e., vector) from which the envelope protein can be expressed in the host cell. However, a plasmid is preferred for this purpose. A suitable expression plasmid may be readily selected by one of skill in the art taking into consideration convenience, the selected expression cells, and the like.

The necessary envelope protein sequences and regulatory elements may be readily engineered into the selected vector. The envelope sequences are readily selected from a variety of sources identified above. The regulatory sequences may be readily selected from among the sequences described above in the section discussing regulatory sequences for the transgene. Thus, the nucleic acid molecule carrying the envelope protein contains the envelope sequences described above under the control of regulatory sequences which direct expression of the envelope protein in a host cell.

Conventional techniques may be utilized for construction of the viral minigenes and other nucleic acid molecules of the invention; See, generally, Sambrook et al, MOLECULAR CLONING: A LABORATORY MANUAL, Cold Spring Harbor Laboratories, Cold Spring Harbor, N.Y. Once the desired vectors are engineered, they may be transferred to a host cell for packaging into the viral envelope by any suitable method.

The host cell itself may be selected from any prokaryotic cell, including any bacterial cell, or any eukaryotic cell, including insects, and yeast, among others. In one desirable embodiment, the host cell is selected from among mammalian species, and particularly from among human cell types. Suitable cells include, without limitation, cells such as CHO, BKH, MDCK, and various murine cells, e.g., 10T1/2 and WEHI cells, African green monkey cells, suitable primate cells, e.g., VERO, COS1, COS7, BSC1, BSC 40, and BMT 10, and human cells such as W138, MRC5, A549, human embryonic retinoblast (HER), human embryonic kidney (HEK), human embryonic lung (HEL), TH1080 cells. Other suitable cells may include NIH3T3 cells (subline of 3T3 cells), HepG2 cells (human liver carcinoma cell line), Saos-2 cells (human osteogenic sarcomas cell line), HuH7 cells or HeLa cells (human carcinoma cell line). In a preferred embodiment, appropriate cells include the human embryonic kidney 293T cells (which express the large T antigen) (ATCC). Neither the selection of the mammalian species providing the cells nor the type of mammalian cell is a limitation of this invention.

Regardless of whether a double transfection or triple transfection technique is utilized, the host cells are cultured according to standard methods. See, e.g., R. J. Wool-Lewis and P. Bates, J. Virol, 74(4):3155-3160 (April 1998); see, also, Sambrook et al, cited above. See, also, Kobinger et al, Nat. Biotech., 19:225-230 (March 2001); and WO 01/83730 (Nov. 8, 2001).

Suitable techniques include cDNA, genomic cloning, which is well known and is described in Sambrook et al, cited above, and use of overlapping oligonucleotides in the target sequences, combined with polymerase chain reaction, synthetic methods, and any other suitable methods which provide the desired nucleotide sequence. Introduction of the molecules (as plasmids or another vector element) into the host cell may also be accomplished using techniques known to the skilled artisan and as discussed throughout the specification. In a preferred embodiment, standard transfection techniques are used, e.g., CaPO₄ transfection, transfection using the Effectene™ reagent, or electroporation, and/or infection by viral vectors into cell lines such as those described above. Each of the desired sequences stably contained within the host cell may be under the control of regulatory elements, such as those discussed above in connection with the transgene. In one particularly suitable embodiment, inducible promoters are selected. For example, for pseudotyped lentiviral production, it may be particularly desirable for gag and pol to be expressed under the control of one or more inducible promoters. However, other suitable regulatory elements may be readily selected by one of skill in the art.

Regardless of the production method utilized, the recombinant viruses of the invention may be readily purified from culture using methods known to those of skill in the art. One suitable method involves ultracentrifugation with or without sucrose or affinity chromatography. Conventional techniques may be used to concentrate the recombinant virus (see, e.g., J. C. Burn et al, Proc. Natl. Acad. Sci. USA, 90:8033-8037 (1993)).

V. Delivery Of Transgenes Via Chimeric Ebola-Pseudotyped Viruses

A. Pharmaceutical Compositions

The chimeric ebola envelope proteins of the invention, recombinant viruses expressing same, and viruses comprising these capsids, can be readily formulated for delivery. Suitably, the chimeric ebola construct of the invention is suspended in a physiologically compatible carrier for administration to a human or non-human mammalian patient. Suitable carriers may be readily selected by one of skill in the art in view of the indication for which the virus is directed. See, e.g., carriers discussed above in connection with the immunogenic compositions. Optionally, the compositions of the invention may contain, in addition to the construct of the invention and carrier(s), other conventional pharmaceutical ingredients, such as preservatives, or chemical stabilizers, such as are described above in connection with the immunogenic compositions.

The recombinant viruses of the present invention may be formulated in such a manner for a variety of uses including in vitro protein and peptide expression, as well as ex vivo and in vivo gene delivery. Thus, the recombinant viruses of the invention may be used to deliver a selected transgene or other molecule to a host cell by any suitable means. In one embodiment, the viruses and the cells are mixed ex vivo and the infected cells are cultured using conventional methodologies. Such methods are described in more detail below. In another embodiment, viruses have been deemed suitable for applications in which delivery of a molecule, e.g., a transgene which permits transient expression, is therapeutic (e.g., p53 gene transfer in cancer and VEGF gene transfer in heart diseases). However, the viruses are not limited to use where transient expression is desired. The viruses are useful for a variety of situations in which delivery of a selected molecule is desired.

B. Transgenes and Delivery Methods

Thus, the invention provides a method of delivering a transgene or other molecule to a human or veterinary patient by transducing the cells of the patient with a recombinant virus with a chimeric ebola according to the invention, optionally in combination with an immunosuppressant or immune suppressing regimen. Alternatively, the chimeric ebola proteins of the invention may be used in a non-viral system for targeting selected molecules to cells having receptors for the ebola binding domain. Suitable molecules may be readily selected from among the transgenes described herein and their products, and from a variety of biologically active compounds, including chemical moieties.

The target cells may be transduced in vivo or ex vivo, taking into consideration such factors as the selection of target cells, the transgene being delivered, and the condition for which the patient is being treated. For example, where the targeted cells are selected from muscle cells, lung cells, liver cells or the like, in vivo transduction may be more desirable. However, where the targeted cells are dendritic cells and/or macrophages, ex vivo transduction is preferred.

1. Therapeutic Products

Useful therapeutic products encoded by the transgene include hormones and growth and differentiation factors including, without limitation, insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), transforming growth factor α (TGFα), platelet-derived growth factor (PDGF), insulin growth factors I and II (IGF-I and IGF-II), any one of the transforming growth factor superfamily, including TGF-α, activins, inhibins, or any of the bone morphogenic proteins (BMP) BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

Other useful transgene products include proteins that regulate the immune system including, without limitation, cytokines and lymphokines such as thrombopoietin (TPO), interleukins (IL) IL-1 through IL-25 (including IL2, IL4, IL12 and IL18), monocyte chemoattractant protein, leukemia inhibitory factor, granulocyte-macrophage colony stimulating factor, Fas ligand, tumor necrosis factors α and β, interferons α, β, and γ, stem cell factor, flk-2/flt3 ligand. Gene products produced by the immune system are also useful in the invention. These include, without limitations, immunoglobulins IgG, IgM, IgA, IgD and IgE, chimeric immunoglobulins, humanized antibodies, single chain antibodies, T cell receptors, chimeric T cell receptors, single chain T cell receptors, class I and class II MHC molecules, as well as engineered immunoglobulins and MHC molecules. Useful gene products also include complement regulatory proteins such as complement regulatory proteins, membrane cofactor protein (MCP), decay accelerating factor (DAF), CR1, CF2 and CD59.

Still other useful gene products include any one of the receptors for the hormones, growth factors, cytokines, lymphokines, regulatory proteins and immune system proteins. The invention encompasses receptors for cholesterol regulation, including the low density lipoprotein (LDL) receptor, high density lipoprotein (HDL) receptor, the very low density lipoprotein (VLDL) receptor, and the scavenger receptor. Other suitable gene products include those useful for lipid regulation, including, e.g., apolipoprotein A (and its isoforms, including ApoAI), apolipoprotein E (and its isoforms, including E2, E3, and E4), ABC1, SRB1, and the scavenger receptor. The invention also encompasses gene products such as members of the steroid hormone receptor superfamily including glucocorticoid receptors and estrogen receptors, Vitamin D receptors and other nuclear receptors. In addition, useful gene products include transcription factors such as jun, fos, max, mad, serum response factor (SRF), AP-1, AP2, myb, MyoD and myogenin, ETS-box containing proteins, TFE3, E2F, ATF1, ATF2, ATF3, ATF4, ZF5, NFAT, CREB, HNF-4, C/EBP, SP1, CCAAT-box binding proteins, interferon regulation factor (IRF-1), Wilms tumor protein, ETS-binding protein, STAT, GATA-box binding proteins, e.g., GATA-3, and the forkhead family of winged helix proteins.

Other useful gene products include, carbamoyl synthetase I, ornithine transcarbamylase, arginosuccinate synthetase, arginosuccinate lyase, arginase, fumarylacetacetate hydrolase, phenylalanine hydroxylase, alpha-1 antitrypsin, glucose-6-phosphatase, porphobilinogen deaminase, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin cDNA sequence. Still other useful products include those useful for treatment of hemophilia A (including Factor VIII and its variants, such as the light chain and heavy chain of the heterodimer and the B-deleted domain; U.S. Pat. No. 6,200,560 and U.S. Pat. No. 6,221,349) and hemophilia B (including Factor IX).

Useful gene products include non-naturally occurring polypeptides, such as chimeric or hybrid polypeptides having a non-naturally occurring amino acid sequence containing insertions, deletions or amino acid substitutions. For example, single-chain engineered immunoglobulins could be useful in certain immunocompromised patients. Other types of non-naturally occurring gene sequences include antisense molecules and catalytic nucleic acids, such as ribozymes, which could be used to reduce overexpression of a target.

Reduction and/or modulation of expression of a gene is particularly desirable for treatment of hyperproliferative conditions characterized by hyperproliferating cells, as are cancers and psoriasis. Target polypeptides include those polypeptides which are produced exclusively or at higher levels in hyperproliferative cells as compared to normal cells. Target antigens include polypeptides encoded by oncogenes such as myb, myc, fyn, and the translocation gene bcr/abl, ras, src, P53, neu, trk and EGRF. In addition to oncogene products as target antigens, target polypeptides for anti-cancer treatments and protective regimens include variable regions of antibodies made by B cell lymphomas and variable regions of T cell receptors of T cell lymphomas which, in some embodiments, are also used as target antigens for autoimmune disease. Other tumor-associated polypeptides can be used as target polypeptides such as polypeptides which are found at higher levels in tumor cells including the polypeptide recognized by monoclonal antibody 17-1A and folate binding polypeptides.

Other suitable therapeutic polypeptides and proteins include those which may be useful for treating individuals suffering from autoimmune diseases and disorders by conferring a broad based protective immune response against targets that are associated with autoimmunity including cell receptors and cells which produce “self”-directed antibodies. T cell-mediated autoimmune diseases include Rheumatoid arthritis (RA), multiple sclerosis (MS), Sjögren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Crohn's disease and ulcerative colitis. Each of these diseases is characterized by T cell receptors (TCRs) that bind to endogenous antigens and initiate the inflammatory cascade associated with autoimmune diseases.

2. Immunogenic Transgenes

In another aspect, the invention provides recombinant viruses with chimeric ebola envelope proteins which contain a transgene encoding a peptide, polypeptide or protein which induces an immune response to a selected immunogen.

For example, immunogens may be selected from a variety of viral families. Example of viral families against which an immune response would be desirable include, the picornavirus family, which includes the genera rhinoviruses, which are responsible for about 50% of cases of the common cold; the genera enteroviruses, which include polioviruses, coxsackieviruses, echoviruses, and human enteroviruses such as hepatitis A virus; and the genera apthoviruses, which are responsible for foot and mouth diseases, primarily in non-human animals. Within the picornavirus family of viruses, target antigens include the VP1, VP2, VP3, VP4, and VPG. Another viral family includes the calcivirus family, which encompasses the Norwalk group of viruses, which are an important causative agent of epidemic gastroenteritis. Still another viral family desirable for use in targeting antigens for inducing immune responses in humans and non-human animals is the togavirus family, which includes the genera alphavirus, which include Sindbis viruses, RossRiver virus, and Venezuelan, Eastern & Western Equine encephalitis, and rubivirus, including Rubella virus. The flaviviridae family includes dengue, yellow fever, Japanese encephalitis, St. Louis encephalitis and tick borne encephalitis viruses. Other target antigens may be generated from the Hepatitis C or the coronavirus family, which includes a number of non-human viruses such as infectious bronchitis virus (poultry), porcine transmissible gastroenteric virus (pig), porcine hemagglutinating encephalomyelitis virus (pig), feline infectious peritonitis virus (cats), feline enteric coronavirus (cat), canine coronavirus (dog), and human respiratory coronaviruses, which may cause the common cold and/or non-A, B or C hepatitis. Additionally, a coronavirus is the putatative causative agent of sudden acute respiratory syndrome (SARS). Within the coronavirus family, target antigens include the E1 (also called M or matrix protein), E2 (also called S or Spike protein), E3 (also called HE or hemagglutin-elterose) glycoprotein (not present in all coronaviruses), or N (nucleocapsid). Still other antigens may be targeted against the rhabdovirus family, which includes the genera vesiculovirus (e.g., Vesicular Stomatitis Virus), and the general lyssavirus (e.g., rabies). Within the rhabdovirus family, suitable antigens may be derived from the G protein or the N protein. The family filoviridae, which includes hemorrhagic fever viruses such as Marburg and Ebola virus may be a suitable source of antigens. The paramyxovirus family includes parainfluenza Virus Type 1, parainfluenza Virus Type 3, bovine parainfluenza Virus Type 3, rubulavirus (mumps virus, parainfluenza Virus Type 2, parainfluenza virus Type 4, Newcastle disease virus (chickens), rinderpest, morbillivirus, which includes measles and canine distemper, and pneumovirus, which includes respiratory syncytial virus. The influenza virus is classified within the family orthomyxovirus and is a suitable source of antigen (e.g., the HA protein, the NI protein). The bunyavirus family includes the genera bunyavirus (California encephalitis, La Crosse), phlebovirus (Rift Valley Fever), hantavirus (puremala is a hemahagin fever virus), nairovirus (Nairobi sheep disease) and various unassigned bungaviruses. The arenavirus family provides a source of antigens against LCM and Lassa fever virus. The reovirus family includes the genera reovirus, rotavirus (which causes acute gastroenteritis in children), orbiviruses, and cultivirus (Colorado Tick fever, Lebombo (humans), equine encephalosis, blue tongue). The retrovirus family includes the sub-family oncorivirinal which encompasses such human and veterinary diseases as feline leukemia virus, HTLVI and HTLVII, lentivirinal (which includes HIV, simian immunodeficiency virus, feline immunodeficiency virus, equine infectious anemia virus, and spumavirinal). The papovavirus family includes the sub-family polyomaviruses (BKU and JCU viruses) and the sub-family papillomavirus (associated with cancers or malignant progression of papilloma). The adenovirus family includes viruses (EX, AD7, ARD, O.B.) which cause respiratory disease and/or enteritis. The parvovirus family feline parvovirus (feline enteritis), feline panleucopeniavirus, canine parvovirus, and porcine parvovirus. The herpesvirus family includes the sub-family alphaherpesvirinae, which encompasses the genera simplexvirus (HSVI, HSVII, varicellovirus (pseudorabies, varicella zoster) and the sub-family betaherpesvirinae, which includes the genera cytomegalovirus (HCMV, muromegalovirus) and the sub-family gammaherpesvirinae, which includes the genera lymphocryptovirus, EBV (Burkitts lymphoma), infectious rhinotracheitis, Marek's disease virus, and rhadinovirus. The poxvirus family includes the sub-family chordopoxyirinae, which encompasses the genera orthopoxvirus (Variola major (Smallpox) and Vaccinia (Cowpox)), parapoxvirus, avipoxvirus, capnpoxyvirus, leporipoxvirus, suipoxvirus, and the sub-family entomopoxyirinae. The hepadnavirus family includes the Hepatitis B virus. One unclassified virus which may be suitable source of antigens is the Hepatitis delta virus. Another virus which is a source of antigens is Nipan Virus. Still other viral sources may include avian infectious bursal disease virus and porcine respiratory and reproductive syndrome virus. The alphavirus family includes equine arteritis virus and various Encephalitis viruses.

The present invention may also encompass immunogens which are useful to immunize a human or non-human animal against other pathogens including bacteria, fungi, parasitic microorganisms or multicellular parasites which infect human and non-human vertebrates, or from a cancer cell or tumor cell. Examples of bacterial pathogens include pathogenic gram-positive cocci include pneumococci; staphylococci (and the toxins produced thereby, e.g., enterotoxin B); and streptococci. Pathogenic gram-negative cocci include meningococcus; gonococcus. Pathogenic enteric gram-negative bacilli include enterobacteriaceae; pseudomonas, acinetobacteria and eikenella; melioidosis; salmonella; shigella; haemophilus; moraxella; H. ducreyi (which causes chancroid); brucella species (brucellosis); Francisella tularensis (which causes tularemia); Yersinia pestis (plague) and other yersinia (pasteurella); streptobacillus moniliformis and spirillum; Gram-positive bacilli include listeria monocytogenes; erysipelothrix rhusiopathiae; Corynebacterium diphtheria (diphtheria); cholera; B. anthracis (anthrax); donovanosis (granuloma inguinale); and bartonellosis. Diseases caused by pathogenic anaerobic bacteria include tetanus; botulism (Clostridum botulinum and its toxin); Clostridium perfringens and its epsilon toxin; other clostridia; tuberculosis; leprosy; and other mycobacteria. Pathogenic spirochetal diseases include syphilis; treponematoses: yaws, pinta and endemic syphilis; and leptospirosis. Other infections caused by higher pathogen bacteria and pathogenic fungi include glanders (Burkholderia mallei); actinomycosis; nocardiosis; cryptococcosis, blastomycosis, histoplasmosis and coccidioidomycosis; candidiasis, aspergillosis, and mucormycosis; sporotrichosis; paracoccidiodomycosis, petriellidiosis, torulopsosis, mycetoma and chromomycosis; and dermatophytosis. Rickettsial infections include Typhus fever, Rocky Mountain spotted fever, Q fever (Coxiella burnetti), and Rickettsialpox. Examples of mycoplasma and chlamydial infections include: mycoplasma pneumoniae; lymphogranuloma venereum; psittacosis; and perinatal chlamydial infections. Pathogenic eukaryotes encompass pathogenic protozoans and helminths and infections produced thereby include: amebiasis; malaria; leishmaniasis; trypanosomiasis; toxoplasmosis; Pneumocystis carinii; Trichans; Toxoplasma gondii; babesiosis; giardiasis; trichinosis; filariasis; schistosomiasis; nematodes; trematodes or flukes; and cestode (tapeworm) infections.

Many of these organisms and/or toxins produced thereby have been identified by the Centers for Disease Control [(CDC), Department of Heath and Human Services, USA], as agents which have potential for use in biological attacks. For example, some of these biological agents, include, Bacillus anthracis (anthrax), Clostridium botulinum and its toxin (botulism), Yersinia pestis (plague), variola major (smallpox), Francisella tularensis (tularemia), and viral hemorrhagic fever, all of which are currently classified as Category A agents; Coxiella burnetti (Q fever); Brucella species (brucellosis), Burkholderia mallei (glanders), Ricimus communis and its toxin (ricin toxin), Clostridium perfringens and its toxin (epsilon toxin), Staphylococcus species and their toxins (enterotoxin B), all of which are currently classified as Category B agents; and Nipan virus and hantaviruses, which are currently classified as Category C agents. In addition, other organisms, which are so classified or differently classified, may be identified and/or used for such a purpose in the future. It will be readily understood that the viral vectors and other constructs described herein are useful to deliver antigens from these organisms, viruses, their toxins or other by-products, which will prevent and/or treat infection or other adverse reactions with these biological agents.

Administration of the vectors of the invention to deliver immunogens against the variable region of the T cells elicit an immune response including CTLs to eliminate those T cells. In RA, several specific variable regions of TCRs which are involved in the disease have been characterized. These TCRs include V-3, V-14, and V-17. Thus, delivery of a nucleic acid sequence that encodes at least one of these polypeptides will elicit an immune response that will target T cells involved in RA. In MS, several specific variable regions of TCRs which are involved in the disease have been characterized. These TCRs include V-7 and V-10. Thus, delivery of a nucleic acid sequence that encodes at least one of these polypeptides will elicit an immune response that will target T cells involved in MS. In scleroderma, several specific variable regions of TCRs which are involved in the disease have been characterized. These TCRs include V-6, V-8, V-14 and V-16, V-3C, V-7, V-14, V-15, V-16, V-28 and V-12. Thus, delivery of a nucleic acid molecule that encodes at least one of these polypeptides will elicit an immune response that will target T cells involved in scleroderma.

3. Ex Vivo

In another embodiment, the viruses of the invention are useful for ex vivo transduction of target cells. Generally, ex vivo therapy involves removal of a population of cells containing the target cells, transduction of the cells in vitro, and then reinfusion of the transduced cells into the human or veterinary patient. Such ex vivo transduction is particularly desirable when the target cells are dendritic cells or macrophages and/or when the transgene or other molecule being delivered is highly toxic, e.g., in the case of some genes used in the treatment of cancer. However, one of skill in the art can readily select ex vivo therapy according to the invention, taking into consideration such factors as the type of target cells to be delivered, the molecule to be delivered, the condition being treated, the condition of the patient, and the like.

In one embodiment it may be desirable to treat a circulating cancer (e.g., leukemia or lymphoma) by ex vivo therapy, by removal of bone marrow cells or peripheral blood T lymphocytes, transduction with the virus of the invention in vitro, and re-infusion of the transduced cells. In another embodiment, it may be desirable to treat a solid tumor by surgical removal of tumor cells, ex vivo transduction of dendritic cells with the transfer virus of the invention carrying an antigenic epitope from the excised tumor, and re-infusing the altered dendritic cells to induce specific immunity to the antigen. In yet another embodiment, it may be desirable to treat hypercholesterolemia or hyperlipidemia by removal of liver cells (hepatocytes), transduction of the cells with a vector carrying the LDLr gene or VLDLr gene in culture, and re-infusion of these cells via the portal vein. Still other suitable conditions for ex vivo therapy and other useful transgenes will be apparent to one of skill in the art.

Generally, when used for ex vivo therapy, the targeted host cells are infected with 10⁵ TU to 10¹⁰ TU transfer viruses for each 10¹ to 10¹⁰ cells in a population of target cells. However, other suitable ex vivo dosing levels may be readily selected by one of skill in the art.

4. In Vivo

For in vivo delivery of the transgenes, any suitable route of administration may be used, including, direct delivery to the target organ, tissue or site, intranasal, inhalation, intravenous, intramuscular, subcutaneous, intradermal, vaginal, rectal, and oral administration. Routes of administration may be combined within the course of repeated therapy or immunization.

Advantageously, the viruses of the invention are particularly well suited to delivery of transgenes and other molecules to lung cells, as these viruses infect from the apical site, and thus are suited to intratracheal, intranasal, aerosol [Penn Century Sprayer Device, Penn Century, Philadelphia, Pa.; U.S. Pat. No. 5,579,578] or other suitable delivery means. Although less desirable, bronchoscopy may also be utilized for delivery. In one particularly desirable embodiment, the virus of the invention is engineered to contain a cystic fibrosis transmembrane conductance regulator (CFTR) gene which is delivered intratracheally. In another embodiment, it may be desirable to treat a solid tumor by injection of a transfer virus carrying a selected transgene (e.g., IL-2, IL-12, TNF, GM-CSF, herpes simplex virus thymidine-kinase (HS-tk), telomerase, a toxic molecule, a suicide gene, or the like), directly into the tumor. However, the invention is not limited as to selection of transgene or other molecule, or route of delivery, as discussed above.

Suitable doses of viruses may be readily determined by one of skill in the art, depending upon the condition being treated, the health, age and weight of the veterinary or human patient, and other related factors. However, generally, a suitable dose may be in the range of 10³ to 10¹⁸, preferably about 10⁵ to 10¹⁶ transducing units (TU) per dose, and most preferably, about 10⁷ to 10⁹ TU for an adult human having a weight of about 80 kg. Transducing Units (TU) represents the number of infectious particles and is determined by evaluation of transgene (e.g., lacZ) expression upon infection of target cells (usually 293T cells) with limiting dilution of each virus preparation. This dose may be formulated in a pharmaceutical composition, as described above (e.g., suspended in about 0.01 mL to about 1 mL of a physiologically compatible carrier) and delivered by any suitable means. The dose may be repeated, as needed or desired, daily, weekly, monthly, or at other selected intervals.

An optional method step involves the co-administration to the patient, either concurrently with, or before or after administration of the viral vector, of a suitable amount of a short acting immune modulator. The selected immune modulator is defined herein as an agent capable of inhibiting the formation of neutralizing antibodies directed against the recombinant vector of this invention or capable of inhibiting cytolytic T lymphocyte (CTL) elimination of the vector. The immune modulator may interfere with the interactions between the T helper subsets (T_{H1} or T_{H2}) and B cells to inhibit neutralizing antibody formation. Alternatively, the immune modulator may inhibit the interaction between T_{H1} cells and CTLs to reduce the occurrence of CTL elimination of the vector. A variety of useful immune modulators and dosages for use of same are disclosed, for example, in Yang et al., J. Virol., 70(9) (September, 1996); International Patent Application No. WO96/12406, published May 2, 1996; and International Patent Application No. PCT/US96/03035, all incorporated herein by reference.

The following examples are provided to illustrate construction and use of the recombinant vectors and compositions of the invention and do not limit the scope thereof. One skilled in the art will appreciate that although specific elements, reagents and conditions are outlined in the following examples, modifications can be made which are meant to be encompassed by the spirit and scope of the invention.

### EXAMPLES

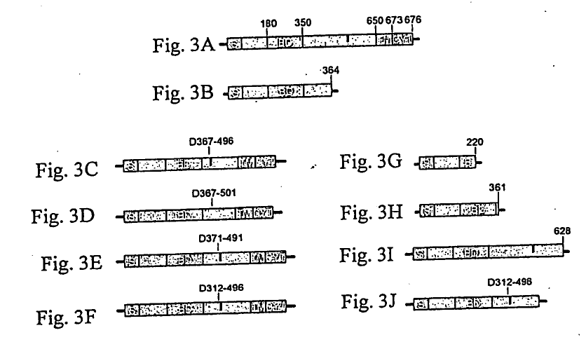

Several chimeric EboZ envelopes were constructed as described in Example 1. In addition, a modified VSV-G with an insertion spanning the putative EboZ GP receptor binding site; and versions of LCMV-GP envelope deleted of internal sequences are described. Examples of hybrid envelopes constructed are illustrated in FIGS. 1A-3J.

Examples 2 to 6 demonstrate testing of exemplary chimeric envelope protein-pseudotyped viral vectors of the invention. More particularly, to determine whether exemplary chimeric EboZ envelope proteins were expressed in cells and incorporated within vector particles, pseudotyped HIV vector particles were produced by triple transfection of 293T cells with an HIV vector, HIV packaging plasmid, and a plasmid expressing one of the modified EboZ envelopes. Vector particles were purified by ultracentrifugation through a sucrose cushion, lysed and used for Western blot analysis. Incorporated envelope was detected by probing with an anti-EboZ polyclonal antibody. A single band corresponding to the expected size (140 kD) was present in all the lysed HIV vector particles pseudotyped with envelopes in which the ectodomain was not altered. The size of the band detected by the anti-EboZ antibody was smaller than EboZ GP for each vector pseudotyped in the NTDL series; the observed mobility of the envelopes corresponded to their predicted size. The hybrid envelopes were incorporated at similar or less amounts as the EboZ GP, but most of the deletion chimera were all incorporated in higher amounts with at least 2-fold greater efficiency than the EboZ GP. Several deletion chimera have been identified which pseudotyped both HIV and FIV vectors resulting in titers up to 5-fold greater than wtEboZ. An FIV vector pseudotyped with a deleted EboZ envelope could be concentrated to high titers and was able to transduce airway epithelia in vivo with high efficiency. Clearly, this vector system is useful for delivering therapeutic genes in vivo to correct the phenotype of genetic diseases.

Examples 7 to 12 are illustrative of the immunogenic and vaccine uses of exemplary chimeric ebo proteins of the invention.

### Example 1

### Construction of Envelopes

The mutations within the EboZ coding region and the development of EboZ/VSV-G hybrid envelopes were performed by PCR site-directed mutagenesis. All envelope constructs were confirmed by sequencing. Plasmids pCB6-EboGP encoding the ebola Zaire (EboZ) envelope [Kobinger G., et al., Nat. Biotech. 2001, cited above] and pMD.G encoding the VSV-G envelope [U. Blomer, et al., J. Virol, 71:6641-6649 (1997)] were used as PCR templates.

The plasmid, pCB-Ebo-GP was constructed using the techniques described in R. J. Wool-Lewis and P. Bates, J. Virol., 74(4):3155-3160 (April 1998). Briefly, the cDNA encoding the Zaire subtype of Ebo-GP was obtained from the Centers for Disease Control and Prevention in the vector pGEM3Zf(−) as a BamHI-KpnI fragment. The Ebo-GP gene was excised from pGEM3Zf(−), using the BamHI and EcoRI restriction enzymes, and cloned into an mammalian expression plasmid pCB6 (purchased commercially) downstream of a human cytomegalovirus promoter to create the plasmid pCB6-Ebo-GP.

For subsequent cloning steps, EboZ restriction enzyme fragments were derived from a pcDNA3.1 plasmid that contains the EboZ coding region with a Kozak consensus translation initiation site. This plasmid was created by amplifying the 5′ region of EboZ in pCB6-EboGP using a sense primer containing the Kozak sequence (AAGGATCCGCCACCATGGGCGTTACAGGAAT, SEQ ID NO:11) and an antisense primer which binds downstream of the ClaI site (GTCACGACAAACTAGTTTT GTCGAC, SEQ ID NO:12). The ClaI/EcoRI fragment from pCB6-EboGP was then used in a triple ligation with the BamHI/ClaI PCR product and the EcoRI/BamHI fragment of the pCDNA3.1 expression vector.

The V/TC, −2aa, +2aa, +16aa, +23aa, V/C and V/2C hybrid envelopes were constructed by amplifying the respective regions of VSV-G using a sense primer that contains EboZ sequences upstream of the junction of the hybrid, and an antisense primer containing an EcoRI site. An antisense primer (GGGAATTCTTACTTTTCCAAGTCGGT, SEQ ID NO:13) was used with the following sense primers for amplification of VSV-G sequences:

| | |
|---|---|
| V/TC, | |
| GGATGGAGAAGCTCTATTGCCTCTTTTTT,; | SEQ ID NO:14 |
| −2aa, | |
| GGATGGAGAATTGCCTCTTTTTTCTTT,; | SEQ ID NO:15 |
| +2aa, | |
| GGATGGAGATGGAAAAGCTCTATTGCC,; | SEQ ID NO:16 |
| +16aa, | |
| GGATGGAGATCCAAAAATCCAATCGAG,; | SEQ ID NO:17 |
| +23aa, | |
| GGATGGAGATTTTTTGGTGATACTGGG,; | SEQ ID NO:18 |
| V/C, | |
| TGTATATGCCGAGTTGGTATCCATCTT,; | SEQ ID NO:19 |
| V/2C, | |
| TTTGTCTTTCGAGTTGGTATCCATCTT,; | SEQ ID NO:20 |

LCMV GP sequences were amplified using:

| | |
|---|---|
| GGATGGAGACTTTTGATGTTTTCAACA, | SEQ ID NQ:21 |
| and | |
| primers. | |
| GGGAATTCTCAGCGTCTTTTCCAGATAG, | SEQ ID NO:22 |

MLV GP sequences were amplified using TGTATATGCCGATTAGTCCAATTTGTTA, SEQ ID NO:23, sense primer with either GGGAATTCTATAGAGCCTGGACCACTGAT, SEQ ID NO:24, or GGGAATTCTATGGCTCGTACTCTATAGGC, SEQ ID NO:25, antisense primers to create the M/C OR M/CR hybrids, respectively.

The EboZ sequence immediately upstream of the junction was also amplified using a sense primer that binds upstream of the BspHI site and an antisense primer that contains VSV-G sequences downstream of the junction of the hybrid. A sense primer (TGGGACCGGACTGCTGT, SEQ ID NO:26) was used with the following antisense primers for amplification of EboZ sequences:

| | |
|---|---|
| V/TC; | |
| AATAGAGCTTCTCCATCCTGTCCACCA,, | SEQ ID NO:27 |
| −2aa; | |
| AGAGGCAATTCTCCATCCTGTCCACCA,, | SEQ ID NO:28 |
| +2aa; | |
| GCTTTTCCATCTCCATCCTGTCCACCA,, | SEQ ID NO:29 |
| +16aa; | |
| ATTTTTGGATCTCCATCCTGTCCACCA,, | SEQ ID NO:30 |
| +23aa; | |
| ACCAAAAAATCTCCATCCTGTCCACCA,, | SEQ ID NO:31 |
| V/C; | |
| ACCAACTCGGCATATACAGAATAAAGCG,, | SEQ ID NO:32 |
| V/2C; | |
| ACCAACTCGAAAGACAAATTTGCATAT,, | SEQ ID NO:33 |
| M/C and M/CR; | |
| GACTAATCGGCATATACAGAATAAAGCG,, | SEQ ID NO:34 |
| L/TC. | |
| CATCAAAAGTCTCCATCCTGTCCACCA,, | SEQ ID NO:35 |

The overlapping PCR products were used as PCR templates, the subsequent PCR products were digested with BspHI/EcoRI, and used in a triple ligation with the BamHI/BspHI pCDNA-EboZ fragment and EcoRI/BamHI fragment of pCDNA3.1. A similar strategy was employed to delete the cytoplasmic domain of EboZ in ΔCyt using the same sense primer that binds upstream of the BspHI site and an antisense primer (TGAATTCCTAGCATATACA-GAATAAAGC, SEQ ID NO:36) that binds to the transmembrane domain and contains an EcoRI site. To create the V/T envelope, the EboZ and plasmid sequences from V/TC envelope excluding the cytoplasmic domain was amplified, and the resulting PCR product religated.

To construct the Δint and Δimm envelopes, VSV-G sequences were amplified using the sense primer TTTTTCATGATTTTTTTGGTGATACTGGGC, SEQ ID NO:37 (ΔInt) or TTTGGCCAACTTTTT TGGTGATACTGGGC, SEQ ID NO:38 (ΔImm) with the antisense primer GGGAATTCTTACTTTCCAAGTC GGT, SEQ ID NO:39. The PCR products were then digested with BspHI/EcoRI (Δint) or MluNI/EcoRI and ligated to the EcoRI/BamHI fragment of pCDNA3.1 and either the BamHI/BspHI or the BamHI/MluNI pCDNA-EboZ fragment.

To create the internally deleted NTDL series of envelopes, PCRs were performed to amplify the regions upstream and downstream of the deletion using overlapping primers. A sense primer (GGACCCGTCTAGTGGCT, SEQ ID NO:40) was used with the following antisense primers to amplify the region upstream of the deletion:

| | |
|---|---|
| NTDL1; | |
| AGTTCTTCTTGTTAGATGCGACACTGCAG,, | SEQ ID NO:41 |
| NTDL2; | |
| AATTGCTTCTGTTAGATGCGACACTGCAG,, | SEQ ID NO:42 |
| NTDL3; | |
| TGTGATCAGTGTGGCAAGGGTTGTTAG,, | SEQ ID NO:43 |
| NTDL4; | |
| CGAGTTCTTCTTACAACTGTGAAAGACAA,, | SEQ ID NO:44 |
| NTDL5; | |
| AGTTCTTCTCTCCCCGATTGTTGTATC,, | SEQ ID NO:45 |
| NTDL6; | |
| AGTTCTTCTGGGGTTGACCTTCCAAAT,, | SEQ ID NO:46 |
| NTDL7; | |
| AGTTCTTCTGCTCCTTTTCCCACTTGT,, | SEQ ID NO:47 |
| NTDL8; | |
| AGTTCTTCTCTCATTCAGCTGGAGCAG,, | SEQ ID NO:48 |
| NTDL9; | |
| AGTTCTTCTGGTCAAATTGTCAACCTC,, | SEQ ID NO:49 |
| and | |
| NTDL10. | |
| AGTTCTTCTTCCAAAACCGGTAGCCTG,, | SEQ ID NO:50 |

An antisense primer (AATAGAGCTTTGTCTCCATCCTGTCCACC, SEQ ID NO:51) was used with the following sense primers to amplify the region downstream of the deletion:

| | |
|---|---|
| NTDL1; | |
| CATCTAACAAGAAGAACTCGAAGAGAA,, | SEQ ID NO:52 |
| NTDL2; | |
| CATCTAACAGAAGCAATTGTCAATGCTC,, | SEQ ID NO:53 |
| NTDL3; | |
| CTTGCCACACTGATCACAGGCGGGAGA,, | SEQ ID NO:54 |
| NTDL4; | |
| CACAGTTGTAAGAAGAACTCGAAGAGAA,, | SEQ ID NO:55 |
| NTDL5; | |
| ATCGGGGAGAGAAGAACTCGAAGAGAA,, | SEQ ID NO:56 |
| NTDL6; | |
| GTCAACCCCAGAAGAACTCGAAGAGAA,, | SEQ ID NO:57 |
| NTDL7; | |
| AAAAGGAGCAGAAGAACTCGAAGAGAA,, | SEQ ID NO:58 |
| NTDL8; | |
| CTGAATGAGAGAAGAACTCGAAGAGAA,, | SEQ ID NO:59 |
| NTDL9; | |
| AATTTGACCAGAAGAACTCGAAGAGAA,, | SEQ ID NO:60 |
| and | |
| NTDL10; | |
| GGTTTTGGAAGAAGAACTCGAAGAGAA,, | SEQ ID NO:61 |

The overlapping PCR products were used as PCR templates, the PCR products were digested with AgeI/BspHI, and used in a triple ligation along with the BspHI/EcoRI fragment of EboZ and the EcoRI/AgeI fragment of pCDNA-EboZ.

To create the internally deleted LCMV, regions of the LCMV GP were amplified using either TTTCTCGAGTTTTCTCACTAGGAGACT, SEQ ID NO:62, or TTTCTCGAGTAGGAGACTTGCAGGCAC, SEQ ID NO:63, sense primers containing an Xho I site (in italics) with GCAACAGCTGTATTCCC, SEQ ID NO:64, antisense primer. The PCR products were then cloned within the Xho I and Pst I sites of the pHCMV-GP (WE-HPI) plasmid expressing the LCMV GP.

Using similar techniques, other constructs of the invention can be generated.

### Example 2

### In Vitro Testing of Chimeric Envelopes

A. FIV Vectors

The pseudotyped FIV vectors were evaluated for production of vector capable of in vitro transduction. Plasmids expressing the envelopes were used for co-transfection of 293T cells with the FIV transfer vector pVC-LacZwP encoding β-gal and the packaging plasmid pCFIVΔorf2Δvif encoding FIV proteins [J C Johnston et al, J Virol, 73:4991-5000 (1999)]. Cotransfections were performed using the CalPhos kit (BD BioSciences Clontech, Palo Alto, Calif., US) with a 2:4:1 ratio of packaging, transfer and envelope plasmids. Cell supernatants containing HIV vector particles were collected 48-72 hrs post transfection and filtered with a 0.45 μm syringe filter. Limiting dilutions were then performed on 293T cells grown in 24-well plates.

Western blots were performed on vector particles purified from cell supernatants by centrifugation onto a 20% sucrose cushion at 200,000×g for 3 hrs at 4° C. Pelleted vector particles were lysed in Laemmli sample buffer and aliquots run in a 4-15% gradient gel (Bio-Rad Laboratories; Hercules). Protein bands were transferred overnight onto a nitrocellulose filter and probed using the ECL-Plus kit (Amersham Pharmacia Biotech; Piscataway N.J., US) with a polyclonal anti-EboZ rabbit antibody and either a monoclonal anti-p26 mouse antibody or a monoclonal anti-p24 human antibody to detect viral capsid proteins. Primary antibodies were detected using horseradish peroxidase-conjugated anti-rabbit, anti-mouse and anti-human goat antibodies (Santa Cruz Biotech; Santa Cruz Calif., US). Bands were visualized by exposure to film (Kodak Biomax MR; Rochester, N.Y., US) and quantified using the AlphaImager system (Alpha Innotech Corporation; US).

The observed mobility of the envelopes corresponded to what was observed for the HIV vector particles. Pseudotyping with the hybrid envelopes resulted in decreased titers compared to EboZ GP envelope. Again, pseudotyping with the NTDL envelopes resulted in increased titers, reaching a peak with NTDL7.

B. HIV Vector

Pseudotyped HIV vector particles were produced and analyzed using the same conditions as for the FIV vector. Cell supernatants from the triple transfections of LacZ vectors were titrated by limiting dilution assays on 293T cells evaluating for LacZ transduction. Envelopes were also used for cotransfection with the HIV transfer vector pHxLacZWP [P F Kelly et al, Ann NY Acad Sci 938:262-276 (2001); G Wang et al, J Clin Invest., 104:R55-62 (1999)] encoding β-gal and packaging plasmid pCMV 8.2 encoding HIV proteins using similar conditions as for the FIV vector.

The HIV titers obtained from pseudotyping with the EboZ/VSV-G hybrid envelopes were 3-4 logs lower compared to the EboZ GP. Vectors pseudotyped with the ΔInt, ΔImm, VE, H/TC, L/TC, ΔL1 and ΔL2 envelopes resulted in negligible to undetectable titers. Pseudotyping with the Ebo/MLV hybrids (M/C, M/CR) resulted in titers in the range of 10³-10⁴ (data not shown). However, pseudotyping with the internally deleted NTDL envelopes resulted in up to 7-fold increase in titer. A corresponding increase in titer was observed the greater the size of the deleted region. The titers reached a maximum value with the NTDL6 envelope for the HIV vector. After this peak, the titers subsequently decreased to the level of the EboZ GP.

Progressive deletions in the EboZ GP from NTDL9 (Δaa241-496 of SEQ ID NO:1) to NTDL10 (Δaa227-496 of SEQ ID NO:1) resulted in very low incorporation of glycoprotein on the vector surface and no detectable transduction in vitro. Sequences critical to EboZ GP protein processing and function were mapped by generating a variety of additional variants. Expanding an internal deletion from aa233-496 (SEQ ID NO:1, NTDL11) to aa232-496 (SEQ ID NO:1, NTDL13) by only one amino acid dropped titers from those observed with EboZ GP to undetectable levels. Amino acid 232 [SEQ ID NO:1] is located at the very end of an alpha helix distal to the variable domain supporting its critical role in envelope assembly or the formation of infectious particles.

### Example 3

### Animal Models

Large scale preparations of FIV and HIV vector particles were obtained by cotransfection of 293T cells using either a 2:4:1 or 1:2:3 ratio of packaging, transfer and envelope plasmids. Cell supernatants were collected 72 hrs post transfection, filtered through a 0.45 μm membrane filter and concentrated by ultracentrifugation at 141,000×g for 2 hrs at 4° C. Tracheas of C57BL/6 mice were exposed by a midline incision and vector particles were injected using a 30 g needle (Becton Dickinson; Franklin Lakes, N.J., US) (HIV-EboZ-lacZ (10⁷), HIV-NTDL4-lacZ (10⁷), FIV-EboZ-lacZ (7×10⁷) and FIV-NTDL4-lacZ (2×10⁸)). After injection, the subcutaneous tissues were sutured closed and the mice maintained in animal facility until necropsy. At day 35 following vector administration, animals were sacrificed and the lungs were inflated with OCT compound (Sakura Finetek; US). Cryosections (10 μm) were prepared and stained with X-gal overnight for lacZ expression.

### Example 4

### Transduction of Pseudotyped Vectors in Human Tracheal Explants

Small pieces (0.5 cm²) are excised from explanted normal or CF human airways and placed on collagen-coated permeable supports. Tissue can be fed from the basolateral surface with media as above. Tissues are infected with 50-100 μl of highly concentrated EboZ or VSV-G-pseudotyped viruses encoding β-galactosidase (β-gal) from the apical surface and incubated for 2-4 h.

Viral titers are determined by limiting dilution on 293T cells. Media is replaced and the tissue is then submerged in media overnight with replacement performed every 12 h. Tissue is fixed in 0.5% glutaraldehyde, stained with X-gal at 37 for 3-12 h, and processed for paraffin embedding. Chimeric EboZ-pseudotyped vector is anticipated to yield staining of the epithelium when incubated in X-gal substrate 24 h post-infection).

### Example 5

### Intratracheal Delivery of HIV- and FIV-Chimeric Ebo

A. Murine Studies

HIV vectors pseudotyped with the EboZ GP envelope have been shown to transduce airway epithelia directly without any requirement for disruption of tight junctions. To determine whether the NTDL envelopes retained this capability of EboZ GP, concentrated HIV and FIV vector particles (1-5×10⁸ TU/ml) pseudotyped with the EboZ GP, the NTDL4 or NTDL6 deletion chimeric were prepared and injected (10⁷ TU/animal) intratracheally into C57Bl/6 mice. Mice were sacrificed at day 35 and airway tissues were stained to visualize β-galactosidase (β-gal) production.

Both the NTDL4- and NTDL6-pseudotyped vectors transduced airway epithelia with high efficiency. In particular, the NTDL6-HIV-lacZ vector gave rise to more transduced cells that had higher expression levels. At this dose, the EboZ GP-pseudotyped vector transduced airway cells with the same efficiency as the NTDL-pseudotyped vectors (data not shown).

The advantage of the NTDL vectors was best illustrated in vivo following the administration of lower doses of vector (10⁶ TU/animal). As expected, only negligible numbers of cells were transduced by vectors pseudotyped with EboZ GP. The HIV and FIV vectors pseudotyped with the NTDL6 envelope transduced significantly more airway cells.

B. Non-Human Primate Study

A panel of EboZ-GP envelope deletion chimera was generated to minimize EboZ-GP associated toxicity and possibly improve vector mediated transduction. The four deletion chimera that showed both reduced cellular toxicity and a significant improvement in transduction efficiency in vitro and, in vivo in immunocompetent mice was tested. HIV-based vector pseudotyped with the Non-Toxic EboZ-GP Deletion 4 or 6 (NTD4 or NTD6) and encoding for the β-galactosidase reporter gene was evaluated following instillation in the lung of non-human primates (rhesus) and compared to the wild-type EboZ-GP envelope pseudotyped HIV vector. Results indicate that HIV-based vector pseudotyped with the Non-Toxic EboZ-GP Deletion chimeric transduce airway epithelia and submucosal glands more efficiently that the wild type EboZ-GP pseudotyped vector. Areas of the transduced lung with pseudotyped HIV vector showed section with up to 18% of β-galactosidase positive cells by histochemical staining. Overall, the instilled lobe contained 1 HIV vector genome copy per 800 total lung cells for NTD4 as opposed to 1 genome per 1000 cells for EboZ-GP pseudotyped HIV vector as determined by Taq-Man PCR. Pseudotyped HIV vector genomes were detected by Taq-Man PCR in the spleen and liver of one rhesus suggesting that vector may spread to internal organs possibly due to airway injury during instillation with the bronchoscope.

Overall, this study shows that deletion chimera of the Ebola envelope are less toxic and that in the context of HIV-based vector and transduction efficiency of non-human primate, airway is improved.

### Example 6

### Production of Recombinant Vector Carrying CFTR Gene

Plasmid HR CFTR (HIV vector containing the CFTR gene) is prepared as follows. CFTR is isolated from AdCBCFTR by a SmaI digestion. Then the SmaI-SmaI CFTR gene is ligated to the pHR backbone in the blunted BamHI/XhoI site by using the Klenow fragment of E. coli.

Plasmid pHR′EFGP is produced as described in WO 01/83730, cited above.

Plasmid RM (CMV/gag-pol/RRE packaging constructs, e.g., without tat, rev, vif, vpr, vpu) is generated as follows. A XhoI site is inserted by PCR mutagenesis at position 4389 in pCMV R8.2 to generate pCMV R8.2XheI (+1 corresponds to the first nucleotide of the HIV sequence after the CMV promoter in pCMV R8.2). Then pCMV R8.2XheI is digested with XhoI/AvaI, which removed all regulatory and accessory genes as well as the envelope, and ligated to 399 nucleotides encompassing the RRE. The XhoI-RRE-AvaI fragment is generated by PCR from pCMV R8.2 with primers: oligo 5′ XheI (sense): SEQ ID NO:65: 5′-AAT TGA ACC ATC TCG AGT AGC ACC C-3′ and oligo 2′ AvaI (anti-sense): SEQ ID NO:66: 5′-CCC ACT CCA TTC COG ACT CGG GAT TCC ACC TGA-3′

Using these constructs, a transfer virus encoding the wt CFTR gene is produced using the methods described above.

### Example 7

### Vaccination with Adenovirus Vectors Expressing Wild Type and Variant EboZ GP

Human serotype 5 adenovirus (AdHu5) or chimpanzee serotype Pan7 adenovirus (AdC7) vectors expressing Ebola envelope chimeras were produced for in vivo immunization experiments in C57BL/6 mice. Five EboZ variants encoded by AdHu5 or AdC7 were selected and produced to evaluate their relative immunogenicity following an intramuscular Ad injection. The wt Ebo, a soluble Ebo variant which contains a truncation as position 364 of the EboZ glycoprotein, EboΔ1, EboΔ2, EboΔ3, EboΔ4, EboΔ5S, EboΔ6S, EboΔ7S and EboΔ8S were evaluated in the initial vaccine studies. The soluble proteins were constructed as described above, for the other variants, and are truncated at the carboxy terminus to remove at least the transmembrane and cytoplasmic domains. See, Figs.

TABLE
Production of Adhu5 or AdC7 Adenoviral
vector encoding EboZ variant.

| | |
|---|---|
| HuAd5^{a} | AdC7^{b} |

| | | Total | | Total |
|---|---|---|---|---|
| | Titer | yield | Titer | yield |
| | (VP × | (VP × | (VP × | (VP × |
| Gene^{a} | 10¹²/ml) | 10¹²) | 10¹²/ml) | 10¹²) |
| Ebo wt | 2.6 | 12 | 4.3 | 43 |
| EboS | 4.9 | 49 | 4.6 | 55 |
| EboΔ2 | 2.1 | 9 | 5.8 | 93 |
| EboΔ3 | 1.7 | 8 | 5.3 | 95 |
| EboΔ4 | 3 | 12 | 4.1 | 62 |

^{a}Number of viral particles (per ml or total) produced and amplified from infected 293 cells as established by spectrophotometry reading.

Vector was administered intramuscularly (10¹¹ genome copies/cell) in C57BL/6 mice and the presence of virus neutralizing antibody (VNA) was evaluated 28 days later as a first measure of an immune response generated against the Ebola envelope glycoprotein. VNA is defined here as serum antibody able to inhibit transduction of HeLa cells mediated by HIV-based vector pseudotyped with the wild-type Ebola envelope.

VNA to the EboZ pseudotypes was detected with AdC7 yielding higher titers than AdHu5. The EboZΔ3 eliciting the highest VNA in terms of the transgene targets (Table).

TABLE
Neutralizing antibody titers to HTV-EboZ-GFP pseudotypes
(reciprocal dilution). N = 5 animals/group.

| | VNA Titers | | |
|---|---|---|---|
| | EboZ wildtype | EboZs | EboZΔ3 |
| AdHu5 | 12 | 16 | 12 |
| AdC7 | 44 | 12 | 140 |

### Example 8

### Study to Evaluate the Immune Response (in Mice) Against Ebola Nuclear Protein and Ebola Envelope Antigens Following Immunization

Mouse studies to evaluate Ebola envelope proteins and the Ebola nuclear antigen were initiated. These studies evaluated neutralizing antibodies in C57Bl/6 mice injected IM with Adhu5 or a chimpanzee adenovirus Pan-7 vector expressing each of 4 Ebola env constructs. The chimpanzee adenovirus Pan-7 vector is the subject of a co-pending, co-owned application.

A. Evaluation of CTL from C57Bl/6 Mice Injected IM with Adhu5 or Pan-7 Expressing the Ebola env Constructs.

1. Challenge Experiment in Mice with Ebola Virus.

Neutralizing antibody (NAB) responses to the Ebola envelope were analyzed by looking at immunized mouse sera mediated neutralization of a lentiviral (HIV) vector pseudotyped with the several constructs (eEbo, NTD2, NTD3, NTD4) of the Ebola envelope glycoprotein. C57BL/6 or BALB/c mice received a single intramuscular injection of 5×10¹⁰ particles per mouse of C7 (Ad Pan-7) encoding Ebola envelope variant. Neutralizing antibody was evaluated 30 days post-vaccination. Briefly, Ebola Zaire pseudotyped HIV vector encoding for β-galactosidase (EboZ-HIV-LacZ) was incubated for 2 hr at 37° C. with different dilution of heat inactivated mouse serum. Following the incubation with serum, EboZ-HIV-LacZ was then used to infect HeLa cells for 16 hr at 37° C. Infectivity was revealed by X-gal staining of transduced HeLa cells positive for β-galactosidase. Neutralizing titer represent the serum reciprocal dilution where a 50% decrease in the number of β-galactosidase positive blue cells was observed.

Sera were collected 30 days post-immunization, which consisted in a single intramuscular (I.M.) administration of 5×10¹⁰ particles/animal. Neutralizing antibody to Ebola pseudotyped HIV could be detected from all groups with antibody titers ranging from 20 for Ad-EboZ (Adhu5 expressing EboZ), Ad-NTDL3 (Adhu5 expressing NTDL3) and C7-sEbo (Ad Pan-7 expressing soluble EboZ) to over 130 for C7-NTD3 (Ad Pan-7 expressing soluble NTDL3) and C7-NTD4 (Ad Pan-7 expressing soluble NTDL3). The same immunization strategy in BALB/c mice resulted in lower neutralizing antibody titers for Ad- and C7-NTDL2, and NTDL4.

B. Cellular Immune Response

The cellular immune response to the Ebola envelope in C57BL/6 mice was evaluated 8 days after a single I.M. administration of 5×10¹⁰ particles of C7-LacZ or C7-Ebola envelope variant per animal. Mice were vaccinated I.M. with 5×10¹⁰ particles of C7 encoding LacZ or Ebola envelope variant. Splenic lymphocytes from immunized mice were collected 8 days post vaccination and stimulated in vitro with feeder cells (splenic lymphocytes from untreated mice infected with human Adenovirus serotype 5 encoding for the wild-type Ebola envelope and irradiated). Standard 5-hr CTL assays were performed using ⁵¹Cr-labeled syngeneic C57 cells transfected with an expresser of EboZ.

A positive MHC-restricted cytotoxic T lymphocyte (CTL) response was observed from all AdPan-7 encoding for Ebola envelope variants with a higher response from NTDL2, NTDL3 or NTDL4 immunized mice. Indeed, effector cells from C7 encoding Ebola envelope variant immunized mice recognized EboZ transfected target cells and gave recall CTL responses up to 30% specific lysis. Less than 5% lysis was seen with effector cells from naïve or LacZ immunized control mice confirming that lysis was specific for Ebola envelope antigens.

C. Protection Studies

The most direct means of evaluating C7 (Ad Pan-7) encoding for the EboZ variants as a successful vaccine in mice was to assess protection against weight loss and death following lethal challenge with mouse adapted Ebola Zaire virus. BALB/c mice were immunized with a single dose of 5×10¹⁰ particles per animal as performed previously and vaccinated animals were challenged with 200 LD₅₀ of mouse adapted Ebola Zaire 21 days later. All control mice (vehicle and C7-LacZ) died between day 5 to day 9 post-challenge. In contrast, all vaccinated mice but one, (from the C7-sEbo group), survived the challenge with Ebola Zaire.

Weight loss was observed from mice vaccinated with C7-sEbo from day 4 to day 7. Signs of illness such as pilo-erection and from light to severe lethargy were also noted from mice vaccinated with C7-sEbo, NTDL2 and NTDL3 between day 4 to day 7. Mice immunized with C7-EboZ and C7-NTDL4 did not show sign of illness. Overall, a single dose of C7-EboZ and C7-NTDL4 completely protected immunized mice from illness and death possibly due to a significant T-cell mediated immunity.

### Example 9

### Additional Studies of Immune Response to Ebola Proteins

Additional studies are performed in which the immunology and efficacy of vaccines expressing the predominant ebola nucleoprotein (NP) are evaluated separately from vaccines expressing optimized glycoprotein (GP). The vaccines will be used at different doses ranging from 10⁷-10¹¹ particles. The vaccine is initially given intramuscularly and where systemic immunization provides protective immunity, additional experiments will deliver the vaccine by the more convenient oral route. The assays for both routes of immunization are similar. Quantitative T cell responses to NP and GP are measured in C57BL/6 mice using an intracellular cytokine assay with peptides to the mapped epitopes. Antibody responses to GP are measured from ICR (outbred), C57BL/6 and C3H/He mice. A surrogate model for protection from Ebola in which Ad-GP/NP vaccinated mice are challenged intraperitoneally with a replicating vaccinia virus expressing the vaccine target (i.e., GP or NP) will be used [Arichi, T., et al., Proc. Natl. Acad. Sci. USA, 97:297-302 (2000)]. Efficacy is read out by measuring the resulting replication of the vaccinia construct in ovaries. A positive result requires a minimum of a 4-log diminution of recovered vaccinia. The time course of the T and B cell responses is carefully evaluated to determine how soon after immunization a detectable response occurs and how long it lasts.

Six constructs (i.e., AdHu5, AdC6 and AdC68 expressing either NP or optimized GP) are evaluated in two additional models. The first evaluates protection from lethal doses of a mouse adapted ebola Zaire (EboZ) strain in C57BL/6 mice. This allows correlation of EboZ protection with the surrogate vaccinia model and immunologic readouts of immunity such as CD8⁺T cell frequencies, CTL activity, and VNA. The second model involves immunization studies of rhesus macaques with measures of CD8⁺T cell frequencies and VNA.

A. Immune Responses to EboZNP and GP in Mice

Vectors based on AdHu5 and/or AdC68 are used to perform pilot experiments for T and B cell assays. For both EboZ NP and GP, CD8+ T cell frequencies are determined by intracellular cytokine analysis; to facilitate the assays, epitopes are mapped. A number of antibody assays are developed against GP. Finally, several structural variants of GP are evaluated for elicitation of VNA to maximize the resulting neutralizing activity.

1. T Cell Responses.

Animals are immunized with adenoviruses expressing EboZ NP or GP and analyzed for a variety of T cell responses including CTL activity by ⁵¹Cr-release assay and intracellular staining for cytokine production by antigen-specific T cells. C57BL/6 and C3H/He mice are injected intramuscularly with 10¹¹ particles of the corresponding adenovirus vaccine. Splenocytes are harvested 7-12 days later and mononuclear cells are evaluated for antigen-specific T cells using the intracellular cytokine assay with individual peptides from NP and GP. Initial studies will be performed without re-stimulation.

The specific T cell epitopes are mapped using the following strategy. Splenocytes are divided into multiple aliquots and each one incubated with an individual peptide from a peptide library to NP or GP together with Brefeldin A for 5 hrs at 37° C. The cells are then washed and incubated with FITC label antibody to mouse CD8 and a Cy-labeled antibody to CD4. After 45 min on ice, cells are washed, and following permeablization, a mixture of PE-labeled antibodies to mouse IFN-γ, IL-2 and TNF-α will be added. Antigen-specific CD8⁺ and CD4⁺T cells are detected by 3-color flow cytometry. The T cell epitopes in the EboZ GP and NP for both C57BL/6 and C3H/He mice are determined.

It should be pointed out that CD4⁺T cell frequencies are generally lower than those of CD8⁺ T cells. Thus, epitopes for this subset may not be defined as readily as for the CD8⁺ T cells that undergo more vigorous expansion upon activation. Previous studies have identified the amino acid sequence TELRTFSI [aa 577-584 of SEQ ID NO:1] from EboZ GP as a CD8⁺T cell epitope in C3H mice [Rao, M. et al., Vaccine, 17:2991-2998 (1999), and the peptide ZYQVNNLEEIC [SEQ ID NO:67] as a dominant epitope for EboZ NP in C57BL/6 mice (unpublished).

For the subsequent experiments, CD8⁺ T cell responses to a given antigen of Ebola will be quantitated by using a pool of those peptides that were shown to carry an epitope (for the mouse haplotype used in the experiment). The same approach will be taken to quantitate CD4⁺T cell responses. This will permit better characterization of the Ebo-specific CD8⁺T cell responses, which are assumed to play a major role in providing protection to challenge. The role of CD4⁺T cells in vaccine-induced protection against Ebola virus infection is less well characterized. The same intracellular cytokine assay used to quantitate CD8⁺T cell responses can evaluate the frequency of antigen specific CD4⁺T cells (simply by adding an antibody to CD4⁺ carrying a 3^{rd} fluorochrome).

It is possible, although not expected, that the frequency of CD4⁺ or CD8⁺T cells is below the limit of detectability (less than 0.1-0.5% of all T cells of a given subset depending on levels of background cytokine production) requiring pre-stimulation before the epitope mapping. As a back-up, the transgene-specific T cells can be expanded by incubating splenocytes in vitro for five days in the presence of a heterologous adenovirus recombinant (i.e., not the same serotype as was used for vaccination) expressing the same transgene at 0.1 pfu-cell. Cells thus expanded will then be re-analyzed by an intracellular cytokine assay as described above.

To confirm that cytokine-producing CD8⁺T cells have lytic activity, splenocytes will be tested in a standard ⁵¹Cr-release assay. Target cells (C47SV for C57Bl/6 effector cells and L929 for C3H/He effectors) are infected for 2 hrs with 10 pfu/cell of a vaccinia virus expressing the protein of interest, labeled for 1 hr with ⁵¹Cr at 37 degrees and washed three times. Target cells (10⁴/well) are co-cultured with various ratios of effector cells in a total volume of 200 μl in V-bottom 96-well microtiter plates. Target cells infected with a vaccinia virus recombinant and effector cells from mice immunized with an adenoviral vector expressing an unrelated transgene product serve as controls. After 5 hrs incubation at 37° C. part of the supernatant are harvested and analyzed in a α-counter to determine percent lysis.

Following identification of the CD8⁺ T cell epitopes, an alternative CTL assay can be used. For C57Bl/6 mice, this assay uses the RMAS cell line which is TAP deficient and thus fails to express stable MHC class I molecules on its surface. Stable expression is achieved upon addition of peptides that bind to MHC class I molecules. For C3H/He mice a similar, although not quite as sensitive, assay can be conducted using peptide-loaded C3H/He mice. For this assay, 5×10⁵ RMAS (or L929) cells are incubated overnight at room temperature in serum-free medium under gentle agitation with 1-5 μg/ml of the epitopic peptides. The next morning, cells are labeled with ⁵¹Cr and then analyzed as target cells for T cell-mediated cytolysis as described above.

Upon oral immunization, T cell frequencies are analyzed both from spleens and from the intraepithelial lymphocyte population of the intestine. This population is harvested as follows. The small intestine is removed from exsanginated mice and placed into medium containing antibiotics. The intestines are transferred onto moist paper towels and connective tissues and Peyers' Patches removed. Each intestine is opened longitudinally and washed repeatedly in Petri dishes containing medium to remove fecal matter. The intestines are cut into 1-2 cm pieces and placed into 125 ml flasks containing a stirring bar and 25 ml medium supplemented with 2% FBS, and antibiotics (gentamycin, streptomycin and ampicillin). Flasks will be incubated for 30 min at 37° C. with gentle stirring. The fluid is given through a tea strainer to remove large debris. The tissue is transferred to 50 ml tubes with 15 ml medium, vortexed for 2 min and again poured through a strainer. This step is repeated. The cell supernatants are pooled, washed and resuspended in 80 ml of medium. Cells are passed through a loosely packed 5 ml glass wool column and resuspended in 8 ml of 40% Percoll. This solution will be layered onto 2 ml of 75% Percoll. Tubes will be centrifuged for 20 min at rt. 600-800 g (2000 rpm). Cells at the interphase between the 40-75% Percoll are collected, washed and used for further experiments. For T cell analysis following oral immunization, antibodies to IL-1 are replaced with those to IL-4 or TGF-β, two cytokines that play a dominant role in mucosal immune responses.

2. B Cell Responses.

The presence of total antibody to Ebola Zaire glycoprotein envelope is determined as follows. The neutralization assay is performed to assess the potential of serum antibody to block transduction of permissive HeLa cells to HV-vector coated (i.e., pseudotyped) with the wild-type Ebola envelope glycoprotein [Kobinger, G P., et al., Nat Biotechnol 19:225-230 (2001)]. Serum samples are obtained by retroorbital bleeding or at termination of the experiment by heart puncture. Sera are heat inactivated at 56° C. for 40 min and stored at −20° C. VNA titers are determined on HeLa cells using an Ebola pseudotyped HIV vector encoding for the β-galactosidase gene product (EboZ-HIV-LacZ) at 1 transducing unit (TU)/cell. Different serum dilutions are incubated with EboZ-HIV-LacZ for 1 hr at 37° C. in a final volume of 100 μl and then use to transduce HeLa cells previously seeded in a 96 well plate. Data are expressed as neutralization titers, which are the reciprocal of the serum dilution resulting in a 50% reduction in the number of transduced cells. Samples are assayed in serial 2-fold dilutions from 1:20 to 1:1280.

Total antibody to Ebola envelope glycoprotein is measured by enzyme-linked immunoadsorbent assay (ELISA). To prepare antigen for ELISA, EboZ-HIV is purified and concentrated by ultracentrifugation (28K for 2 hr at 4° C. in a Beckman SW28 rotor). Microtiter plates (Dynatech, Vienna, Va.) or high-binding RIA/EIA flat bottom plates (Costar) are coated with 50 μl/well of antigen at a predetermined optimal dilution (1:500 or 1:1000) in PBS. Plates are incubated at 4° C. overnight and then nonspecific binding was blocked by the addition of 200 μl/well of 5% powdered nonfat milk in PBS containing 0.02% Tween 20. Test antibodies are diluted in either half-log or fivefold increments. Secondary antibody is horseradish peroxidase (HRPO)-labeled goat anti-mouse IgG antibody and the detector substrates are either 2,2′-azino-di 3-ethybenthiazoline sulfonate or 3,3′, 5,5′,-tetramethylbenzidine (TMB, Kirkgaard and Perry Laboratories). Values are read at 405 nm (ABTS) or 450 nm (TMB). Endpoint titers are defined at OD values>0.2 above the mean OD value obtained with the same dilution of serum from control mice. Isotypes of the antibodies to EboZ are tested using the same ELISA with the Calbiochem Hybridoma Subisotyping (LaJolla, Calif.) kit with some minor previously described modifications [Xiang, Z., et al, J. Virol., 76:2667-2675 (2002)]. Sera is tested at a 1:100 dilution.

The final B cell assay is based on a B cell ELISPOT. Multi screen plates will be screened with purified EboZ GP pseudotyped HIV vectors. Following a series of washes, the plates are incubated with 10⁵ to 2×10⁶ lymphocytes per well. Cells will be isolated from spleens and placed in wells at a density of 10⁵ to 2×10⁶ cells per well for a duration of four hours. Plates are then washed and spots reflecting antibodies of different subtypes will be visualized using a Calbiochem Hybridoma subisotyping kit.

Upon oral immunization, antibody titers and antibody isotype distribution from vaginal lavage fluids (which can be harvested without sacrificing the animals) is analyzed. Upon euthanasia, B cell frequency is determined from spleen as well as from the interstitial lymphocyte population of the lungs, which will be recovered as follows. Lungs will be collected from exsanguinated mice and transferred to Petri dishes and washed with PBS. Lungs are cut into small pieces and digested in serum-free medium with 50 U/ml of collagenase I, and 66 U/ml RNAse free Dnase 1 for 60 min at 37° C. Thereafter, cells are shaken vigorously and large clumps will be allowed to settle for 1-2 min. Cells are transferred into a 15 ml tube. ½ volume of 100% Percoll is added and tubes are vortexed. Cells are underlaid with 2 ml of 70% Percoll and centrifuged for 10 min at 2000 rpm. Cells at the interphase are harvested, washed twice and tested in the ELISPOT assay.

### Example 10

### Protective Immunity to EboZ with Novel Simian Adenovirus Vectors in Mice

A. Characterization of B and T Cell Responses

Each novel adenoviral vaccine is characterized in a number of varying strains. Analysis of T cells is performed in C57BL/6 and C3H/He mice utilizing mapped CD8⁺T cell epitopes. Antibody responses are assessed in these strains as well as the outbred ICR mice which better mimic the genetic diversity seen in human populations. Systemic immunity is achieved by intramuscular injection of the adenoviral vector at doses ranging from 10⁷ particles to 10¹¹ particles per animal. Experiments are performed with constructs that independently express NP and optimized GP. Each group contains 10 animals per time point. The initial studies harvest splenocytes at 10 days and mononuclear cells and are analyzed for T cell frequencies, cytolytic activity using standard Chromium release assays. Both T cell frequency and CTL analysis are performed from cells that have not been restimulated to provide a more quantitative assessment of T cell activation in vivo. If the CD8⁺T cell frequencies and CTL activity are undetectable, then restimulation is achieved using a heterologous E1-deleted adenovirus vector expressing the same transgene product or peptides to the mapped epitopes.

1. Characterization of B Cell Response.

C57BL/6, C3H and ICR mice are immunized with the corresponding adenovirus vaccines expressing optimized GP as described above. Serum and splenocytes will be harvested at day 28 for assessment of B cell activation to the EboZ GP. Analysis of GP specific antibodies will utilize the neutralizing assay with pseudotyped lentiviral vector as well as a measure of total anti-EboZ GP using an ELISA assay. The frequency of antigen specific B cells will be determined by ELISPOT in cells harvested from spleen. The distribution of Ig isotypes and the expressing B cells are determined by the ELISA and ELISPOT assays.

2. Vaccinia Virus as a Surrogate Model for Protection.

This strategy is based on immunization of mice with adenovirus expressing the EboZ antigens and subsequent intraperitoneal challenge with a replicating vaccinia virus that expresses the same transgene product. Vaccinia virus recombinants based on the Copenhagen strain of vaccinia virus are constructed with the Ebo inserts provided following previously described procedures [He, Z., et al., Virology, 270:146-171 (2000)]. The recombinants are expanded, titrated in Tk⁻ cells, followed by testing for expression of the Ebo sequences by Western blot or immunoprecipitation.

CTL activity elicited as a result of the vaccine facilitates the clearance of vaccinia virus from the peritoneal cavity. Groups of ten vaccinated or sham-vaccinated animals will be challenged intraperitoneally with 10⁶ pfu of the vaccinia virus recombinant expressing the transgene used in the vaccine. Five days later, ovaries are harvested, homogenized, freeze-thawed three times, and the cell-free homogenates are titrated in TK⁻ monolayers in 24 well plates as described previously. The expected degree of protection, as determined by vaccinia virus titers in ovaries, is expected to correlate with frequency of CD8⁺ T cells to the Ebola antigen. Protective T cell responses should lead to at least a 4-log reduction in quantity of recovered vaccinia virus. Splenocytes of vaccinia virus-challenged mice are tested for frequency of CD8⁺T cells to the Ebola antigen to gain information on the effect of a heterologous boost on the frequency of this T cell population. The result of this surrogate challenge model guides the challenge with the mouse-adapted EboZ virus. These studies are restricted to vaccines and vaccine doses that provide adequate protection against vaccinia virus challenge (reduction of vaccinia virus titers in ovaries>4 logs).

3. Protection from Challenge with a Mouse-Adapted Strain of EboZ.

A murine model of EboZ infection has been developed through the isolation of a strain of EboZ that has undergone serial passage in the mouse [Bray, M., et al., J Infect Dis, 178:651-661(1998); Bray, M. (2001), J Gen Virol, 82:1365-1373]. The mouse-adapted EboZ causes a lethal syndrome with some features similar to that observed in primates. Characterization of this mouse-adapted strain of EboZ revealed only 8 amino acid differences with the wild type parental strain [Volchkov, V. E., et al., Virol, 277:147-155 (2000)]. Challenge of guinea pigs and rhesus monkeys with this strain of virus leads to lethal consequences, further confirming its utility as an authentic model.

In order to evaluate protection, groups of 15 mice are vaccinated intramuscularly with the corresponding simian adenovirus vector expressing either NP or optimized GP. Vector is administered at doses equal to or above those shown to protect against the surrogate challenge. One month later, mice are transferred into the containment area and challenged by intraperitoneal inoculation with 1000 pfu of mouse-adapted Ebola virus (approximately 30,000 times the dose lethal for 50% of adult mice). In order to measure viral titers, five animals from each group are harvested on day 5 and titers of virus are determined from serum using an Ebola virus plaque assay. The other 10 mice are observed for the development of disease. The expectation is that 100% of the non-vaccinated animals will die within 7 to 14 days. All surviving animals will be necropsied at day 28.

A number of parameters will be evaluated in the context of these challenge experiments. Initial studies will investigate the protection afforded by adenoviruses expressing either NP or optimized GP at varying doses. Additional experiments will be performed with equal mixtures of the same serotype adenovirus containing both NP and optimized GP vectors (10⁹, 10¹⁰ and 10¹¹ particles per vector).

4. Time Course of Response.

The time course of T and B cell activation following a single administration of an adenovirus vaccine will be important in assessing both the duration of protection as well as the speed with which protection can be achieved. The latter point is particularly important when considering this vector system as a means to diminish mortality following an attack with the agent and to diminish secondary infection and spread of the agent. Initial studies utilize C57BL/6 mice immunized intramuscularly and harvested at days 3, 6, 9, 12, 15, 21 and 28 (5 animals per group). Serum and spleen are recovered for antibody and T cell assays. The quantitative measures of T and B cell function are correlated with the response of the animals to intraperitoneal challenge with vaccinia virus which are delivered 1, 2, 4, 7, 14, and 28 days after the vaccine. A subset of time points is analyzed in mice in terms of protection against the murine-adapted strain of EboZ. These experiments address two strategic uses of the vaccine. The first is to quickly immunize a population that is at acute risk such as noninfected individuals in close contact to those who were primarily infected from an attack. In this scenario, animals are challenged 1, 2, 4, 7, 14, and 28 days after the vaccine. The second application is to see if vaccination after exposure minimizes morbidity and mortality. For these experiments, animals are vaccinated 1, 2, 3, 4, and 7 days after challenge with the mouse adopted strain of EboZ. The dose of challenge virus varies from 1000, 100, and 10 pfu. It is hoped that these experiments define the time frame following adenovirus in which a protective response can be assured.

The second issue with respect to the time course of immune activation relates to the production of memory B and T cells. This is assessed in animals immunized and subsequently analyzed at later time points including 3, 6, 9 and 12 months after immunization. In each case, the animals are evaluated for B and T cell responses, clearance of intraperitoneal vaccinia virus infection and protection against mouse-adapted EboZ.

5. Mechanisms of Protection

The advantage of working in the mouse system is that it provides an opportunity to better define mechanisms of protection. The following experiments are performed in the appropriate challenge models.

The surrogate protection model of vaccinia virus clearance is primarily based on activation of CTL, which limits its utility in further evaluating the relative contribution of cellular versus humoral immunity. However, a model of protection against the murine-adapted strain of EboZ is very useful in addressing some of these issues. Initial experiments are designed to evaluate the relative contribution of T cell responses versus antibody responses. Questions relevant to protection following vaccination with NP center around the role of CD4⁺ versus CD8⁺ T cells. This can be better defined by depleting either the CD4⁺ or CD8⁺ T cell fraction immediately prior to challenge. In groups of 10 animals, mice are immunized and 28 days later treated with either three doses of GK 1.5 antibody which eliminates CD4⁺ T cells, 53.6.7 antibody which eliminates CD8⁺ T cells, or a combination of both. A control group receives similar quantities of Ig preparation to an irrelevant antigen. Antibodies are injected intraperitoneally three times in a 48-hour interval. Flow cytometry is performed on peripheral blood lymphocytes the day after completion of the regimen to assure depletion of peripheral blood CD4⁺ and CD8⁺ T cells. One day following the last injection of depleting antibody, animals are relocated and challenged with the mouse-adapted EboZ strain. Protection afforded by adenovirus expressing optimized GP could be provided through T cell or B cell mechanisms. Similar studies of CD4³⁰ and CD8⁺ T cells are performed as described for the NP production studies. To evaluate the contribution of neutralizing antibodies to the protective effect, a separate set of animals receives a prime boost regimen of the adenovirus expressing optimized GP using different serotypes in order to boost the level of VNA. Serum from these animals is obtained and passively transferred into recipient non-immunized animals immediately prior to challenge. In this experiment, control mice receive an equal dose of serum from mice immunized with the same vaccine carrier expressing an unrelated antigen.

### Example 11

### Protective Immunity to EboZ in Nonhuman Primates

During the course of the studies described above, a number of vaccine targets (e.g., NP and optimized GP) are evaluation for induction of immune responses in rodents and protection following challenge in the vaccinia virus model and the mouse-adapted EboZ model

Guinea pigs of the inbred strain 13 have been shown to be sensitive to an adapted strain of EboZ demonstrating a substantial viremia, and death due to multi-organ damage between days 8 and 11 following challenge [Ryabchikova, E. I., et al, Curr Top Microbiol Immunol, 235:145-173 (1999); Connolly, B. M., et al, J Infect Dis 179 Suppl 1: S203-217 (1999)]. The purpose of the guinea pig studies is to demonstrate some level of efficacy before proceeding to primate studies because the guinea pig model appears more discerning than the mouse challenge model. Animals will be immunized with various doses of the adenovirus vaccine (10⁹, 10¹⁰ and 10¹¹ particles per injection) and 28 days later challenged with the guinea pig-adapted strain of EboZ. Specifically, the animals are injected subcutaneously with 1000 LD₅₀ (10⁴ pfu) of guinea pig-adapted Mayingh strain Ebola virus. The animals are observed for 28 days and blood is obtained at days 7, 14 and 21 for assessment of viremia using a plaque assay (N=2 per time point). Each group contains 12 animals, with a control group receiving a similar dose of the adenovirus expressing an irrelevant gene.

The following experiments will be conducted in rhesus macaques. In each case an experimental group includes 4 animals with 1-2 control animals that receive similar doses of the recombinant virus expressing an irrelevant gene. The vaccine is injected intramuscularly into vasus lateralis in a volume of 1 ml over 2 sites. For all experiments, animals are vaccinated and evaluated for T and B cell responses to the vaccine target prior to consideration for challenge experiments. Briefly, two base-line blood samples are obtained prior to vaccination for assessment of CD8⁺ T cell frequencies and VNA. Similar samples will be derived 10, 20, 30, 60 and 90 days after the vaccine, at which time determination is made whether to consider the animals for challenge.

The initial primate experiments evaluate the relative performance of the vectors with the optimized EboZ GP as a target at an intramuscular dose of 10¹² particles. The vector providing the highest level of protection will be considered the lead candidate. The next set of experiments evaluates the efficacy of different vaccine targets in the context of the lead vector. The three experimental groups include GP alone, NP alone and GP with NP together, all at a dose of 10¹² delivered intramuscularly. Currently, the GP/NP based vaccine and will do so unless it is very clear that either GP or NP alone provide little evidence of immunologic and/of clinical efficacy. Thereafter, efficacy is studied at 10¹⁰, 10¹¹, and 10¹² particles. The final experiment evaluates the ability of the best EboZ based vaccine to cross protect against lethal challenges of ebola Sudan (EboS) and ebola Ivory Coast (EboIC).

### Example 12

### Cross Protection of an Ebola Vaccine

To address the issue of cross protection, the vaccinia surrogate challenge model is used. Vaccinia viruses are generated expressing EboS and EboIC GP and NP in much the same way they were developed for EboZ genes. Mice are immunized with the lead adenoviral vector expressing EboZ GP and/or NP. The animals are challenged with vaccinia viruses expressing one of the following genes: EboZ GP, EboZ NP, EboS GP, EboS NP, EboIC GP, and EboIC NP intraperitoneally as described [Xiang, Z., J Virol., 76:2667-2675 (2002)]. Protection is considered when the amount of vaccinia virus recovered from homogenates of ovaries is diminished at least 4 logs as a result of the vaccination.

Rhesus macaques are evaluated for cross protection as well. Animals are vaccinated with a control virus or the lead vector candidate expressing EboZ derived NP and GP. Each group of control and vaccinated animals will be challenged with lethal doses of clinical isolates of EboZ, EboS or EboIC.

All publications cited in this specification are incorporated herein by reference. While the invention has been described with reference to a particularly preferred embodiment, it will be appreciated that modifications can be made without departing from the spirit of the invention. Such modifications are intended to fall within the scope of the appended claims.

1. A chimeric ebola envelope protein comprising a functional ebola glycoprotein binding domain fused to a heterologous amino acid sequence, wherein said chimeric protein comprises a functional deletion in an ebola envelope protein between a signal peptide and a cytoplasmic domain.

2. The chimeric ebola envelope protein according to claim 1, wherein said protein contains a wild-type ebola glycoprotein binding domain.

3. The chimeric ebola envelope protein according to claim 1, wherein said heterologous amino acid sequence is an ebola glycoprotein sequence which is non-contiguous with the binding domain in the wild-type ebola.

4. The chimeric ebola envelope protein according to claim 1, wherein said chimeric protein comprises an ebola signal peptide and an ebola binding domain having a deletion in the native ebola region between the signal peptide and the binding domain.

5. The chimeric ebola envelope protein according to claim 4, wherein said chimeric protein comprises a deletion of about 1 to 50 amino acids between the signal peptide and the binding domain.

6-16. (canceled)

17. The chimeric ebola envelope protein according to claim 1, selected from the group consisting of:
(a) NTDL1, amino acids 1 to 366 fused to amino acids 497 to 676 of the ebola glycoprotein, SEQ ID NO:1;
(b) NTDL2, amino acids 1 to 366 fused to amino acids 502 to 676 of the ebola glycoprotein. SEQ ID NO:1;
(c) NTDL3, amino acids 1 to 370 fused to amino acids 492 to 676 of the ebola glycoprotein, SEQ ID NO:1;
(d) NTDL4, amino acids 1 to 311 fused to amino acids 497 to 676 of the ebola glycoprotein, SEQ ID NO:1;
(e) NTLD5, amino acids 1 to 287 fused to amino acids 497 to 676 of the ebola glycoprotein, SEQ ID NO:1;
(f) NTDL6, amino acids 1 to 279 fused to amino acids 497 to 676 of the ebola glycoprotein, SEQ ID NO:1;
(g) NTDL7, amino acids 1 to 267 fused to amino acids 497 to 676 of the ebola glycoprotein, SEQ ID NO:1;
(h) NTDL8, amino acids 1 to 258 fused to amino acids 497 to 676 of the ebola glycoprotein, SEQ ID NO:1;
(i) NTDL9, amino acids 1 to 232 fused to amino acids 497 to 676 of the ebola glycoprotein, SEQ ID NO:1;
(j) NTDL11, amino acids 1 to 231 fused to amino acids 497 to 676 of the ebola glycoprotein, SEQ ID NO:1;
(k) ΔN, amino acids 1 to 31 fused to 172 to 676 of the ebola glycoprotein, SEQ ID NO:1;
(l) EboΔ5S, amino acids 1 to 220 of the ebola glycoprotein, SEQ ID NO:2;
(m) EboΔ6S, amino acids 1 to 361 of the ebola glycoprotein, SEQ ID NO:2;
(n) EboΔ7S, amino acids 1 to 628 of the ebola glycoprotein, SEQ ID NO:2; and
(o) EboΔ8S, amino acids 1 to 311 fused to amino acids 497 to 664 of the ebola glycoprotein, SEQ ID NO:2;
(p) V/TC, amino acids 1 to 672 of SEQ ID NO:1 fused to amino acids 463 to 511 of SEQ ID NO:3;
(q) −2aa, amino acids 1 to 672 of SEQ ID NO:1 fused to amino acids 465 to 511 of SEQ ID NO:3;
(r) +2aa, amino acids 1 to 672 of SEQ ID NO:1 fused to amino acids 461 to 511 of SEQ ID NO:3;
(s) +16aa, amino acids 1 to 672 of SEQ ID NO:1 fused amino acids 447 to 511 of SEQ ID NO:3;
(t) +23aa, amino acids 1 to 672 of SEQ ID NO:1 fused to amino acids 440 to 511 of SEQ ID NO:3;
(u) +42aa, amino acids 1 to 672 of SEQ ID NO:1 fused to amino acids 421 to 511 of SEQ ID NO:3;
(v) V/C, amino acids 1 to 672 of SEQ ID NO:1 fused to amino acids 483 to 511 of SEQ ID NO:3;
(w) V/T, amino acids 1 to 650 of SEQ ID NO:1 fused to amino acids 463 to 482 of SEQ ID NO:3;
(x) ΔInt, amino acids 1 to 629 of SEQ ID NO:1 fused to amino acids sequences 463 to 511 of SEQ ID NO:3;
(y) ΔImm, amino acids 1 to 563 of SEQ ID NO:1 fused to amino acids 463 to 511 of SEQ ID NO:3;
(z) VE, amino acids 180 to 350 of SEQ ID NO:1 in the VSV-G envelope, SEQ ID NO:3.
(aa) H/TC, amino acids 1 to 650 of SEQ ID NO:1 fused to amino acids 661 to 856, SEQ ID NO:8;
(ab) M/C, amino acids 1 to 650 of SEQ ID NO:1 fused to a VSV-G transmembrane domain, 465 to 482 of SEQ ID NO:3, and an MLV-GP cytoplasmic domain, amino acids 634 to 649 of SEQ ID NO:6;
(ac) M/CR, amino acids 1 to 650 of SEQ ID NO:1 fused to a VSV-G transmembrane domain, 465 to 482 of SEQ ID NO:3, an MLV-GP cytoplasmic domain, amino acids 634 to 649 of SEQ ID NO:6, and an R peptide of MLV-GP, amino acids 650 to 665 of MLV-GP, SEQ ID NO:6;
(ad) L/TC, amino acids 1 to 650 of SEQ ID NO:1, fused to amino acids 439 to 498 of LCMV-GP, SEQ ID NO:9.

18-48. (canceled)

49. The chimeric ebola envelope protein according to claim 1, wherein said chimeric comprises a deletion of the complete ebola signal peptide or a portion thereof.

50. The chimeric ebola envelope protein according to claim 1, wherein said deletion of all or a portion of the carboxy terminus of the signal peptide comprises a deletion of from about 1 to 30 amino acids.

51. The chimeric ebola envelope protein according to claim 1, wherein said chimeric ebola envelope comprises a deletion of all or a portion of the ebola transmembrane.

52. The chimeric ebola envelope protein according to claim 51, wherein the deletion of the ebola transmembrane comprises deletion of about 1 to 23 amino acids.

53. The chimeric ebola envelope protein according to claim 1, wherein said chimeric ebola envelope comprises a deletion of all or a portion of the ebola cytoplasmic domain.

54. The chimeric ebola envelope protein according to claim 53, wherein the deletion of the ebola cytoplasmic domain comprises about 1 to 3 amino acids.

55. The chimeric ebola envelope protein according to claim 1, wherein said chimeric ebola envelope comprises a transmembrane domain.

56. The chimeric ebola envelope protein according to claim 55, wherein the transmembrane domain is from a heterologous protein.

57. The chimeric ebola envelope protein according to claim 1, wherein said protein further comprises a cytoplasmic domain.

58. The chimeric ebola envelope protein according to claim 1, wherein said heterologous amino acid sequence is from a non-ebola protein.

59. The chimeric ebola envelope protein according to claim 58, wherein the heterologous amino acid sequence is selected from the group consisting of a Vesicular Stomatitis Virus protein; a human immunodeficiency virus transmembrane domain; a murine leukemia virus; and a Lymphocytic Choriomeningitis virus.

60. A nucleic acid molecule encoding a chimeric ebola protein according to claim 1.

61. The molecule according to claim 60, wherein said molecule is a plasmid.

62. The molecule according to claim 60, wherein said molecule is a viral vector.

63. The molecule according to claim 60, wherein said molecule is an adenoviral vector.

64. A host cell comprising a protein according to claim 1.

65. A host cell comprising a molecule according to claim 60.

66. A method of inducing an immune response against ebola comprising the step of delivering to a subject a composition comprising a protein according to claim 1.

67. The method according to claim 66, wherein said composition is delivered intramuscularly.

68. The method according to claim 66, wherein said composition is delivered orally.

69. A method of inducing an immune response against ebola comprising the step of delivering to a subject a composition comprising a molecule according to claim 60.

70. The method according to claim 69, wherein said composition is delivered intramuscularly.

71. The method according to claim 69, wherein said composition is delivered orally.

72. A recombinant virus having a chimeric ebola envelope protein according to claim 1 and a minigene.

73. The recombinant virus according to claim 72, wherein said minigene is a lentivirus minigene comprising Rev response element (RRE) sequences.

74. The recombinant virus according to claim 72, wherein said lentivirus sequences are selected from the group consisting of a human immunodeficiency virus (HIV) vector, simian immunodeficiency virus (SIV) vector, caprine arthritis and encephalitis virus, equine infectious anemia virus, visna virus, and feline immunodeficiency virus (FIV) vector.

75. The recombinant virus according to claim 74, wherein said lentivirus is an HIV.

76. The recombinant virus according to claim 74, wherein said 5′ LTR sequences are self-inactivating.

77. The recombinant virus according to claim 76, wherein said 5′ LTR sequences contain a deletion in the U3 region.

78. The recombinant virus according to claim 74, wherein said 3′ LTR sequences are self-inactivating.

79. The recombinant virus according to claim 78, wherein said 3′ LTR sequences contain a deletion in the U3 region.

80. A host cell containing a recombinant virus according to claim 72.

81. A method of treating a patient with a selected molecule, said method comprising the step of transducing the cells of the patient with the recombinant virus according to claim 72.

82. The method according to claim 81, wherein the cells are selected from among the lung cells, dendritic cells and macrophages.

83. The method according to claim 81, wherein said recombinant virus is administered directly to the patient.

84. The method according to claim 82, wherein the transgene is a CFTR gene and said recombinant virus is administered intratracheally.

85. The method according to claim 81, wherein the cells of the patient are transduced ex vivo, further comprising the step of re-infusing the transduced cells into the patient.

86. The method according to claim 85, wherein the patient is a cancer patient.

87. The method according to claim 85, wherein the transduced cells are dendritic cells.

88. The method according to claim 85, wherein the transduced cells are macrophages.

89. A method of delivering a molecule to the apical cells of the lung, said method comprising the step of administering a recombinant virus according to any of claims 72 intratracheally.

90. An immunogenic composition comprising a DNA molecule encoding a chimeric ebola envelope protein according to claim 1 under the control of sequences which direct expression thereof in a host cell and a carrier.

91. The immunogenic composition according to claim 90 comprising a recombinant virus comprising the DNA molecule.

92. An immunogenic composition comprising an ebola envelope protein and a carrier, wherein said composition comprises an ebola envelope protein according to claim 1.

93. The immunogenic composition according to claim 92, wherein the immunogenic composition further comprises a wild-type ebola G or S protein.


SEQUENCE LISTING (67 sequences)

SEQ ID NO: 1 - protein, 676 aa, Ebola virus
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 2 - protein, 364 aa, Ebola virus
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKTSLEKFAVKSCLSQLYQTEPKTSVVRVRRELLPTQGPTQQLKTTKSWLQKIPLQWFKCTVKEGKLQCRI

SEQ ID NO: 3 - protein, 511 aa, Vesicular stomatitis virus
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 4 - DNA, 147 bp, Vesicular stomatitis virus
agctctattgcctcttttttctttatcatagggttaatcattggactattcttggttctccgagttggtatccatctttgcattaaattaaagcacaccaagaaaagacagatttatacagacatagagatgaaccgacttggaaag

SEQ ID NO: 5 - DNA, 126 bp, Vesicular stomatitis virus
ttcgaacatcctcacattcaagacgctgcttcgcaacttcctgatgatgagagtttattttttggtgatactgggctatccaaaaatccaatcgagcttgtagaaggttggttcagtagttggaaa

SEQ ID NO: 6 - protein, 654 aa, Murine leukemia virus
MARSTLSKPPKDKINPWKSLMVMGVLLRVGMAESPHQVFNVTWRVTNLMTGRTANATSLLGTVQDAFPRLYFDLCDLVGEEWDPSDQEPYVGYGCKYPAGRQRTRTFDFYVCPGHTVKSGCGGPGEGYCGKWGCETTGQAYWKPTSSWDLISLKRGNTPWDTGCSKVACGPCYDLSKVSNSFQGATRGGRCNPLVLEFTDAGKKANWDGPKSWGLRLYRTGTDPITMFSLTRQVLNVGPRVPIGPNPVLPDQRLPSSPVEAIPAPQPPSPLNTSYPPSTASTPSTSPTSPSAPQPPPGTGDRLLALVKGAYQALNLTNPDKTQECWLCLVSGPPYYEGVAVVGTYTNHSTAPANCTATSQHKLTLSEVTGQGLCMGAVPKTHQALCNTTQSAGSGSYYLAAPTGTMWACSTGLTPCLSTTVLNLTTDYCVLVELWPRVIYHSPDYMYGQLEQRTKYKREPVSLTLALLLGGLTMGGIAAGIGTGTTALIKTQQFEQLHAAIQTDLNEVEKSITNLEKSLTSLSEVVLQNRRGLDLLFLKEGGLCAALKEECCFYADHTGLVRDSMAKLRERLNQRQKLFESGQGWFEGLFNRSPWFTTLISTIMGPLIVLLLILLFGPCILNRLVQFVKDRISVVQALVLTQQYHQLKPIEYEP

SEQ ID NO: 7 - DNA, 96 bp, Murine leukemia virus
cgattagtccaatttgttaaagacaggatatcagtggtccaggctctagttttgactcaacaatatcaccagctgaagcctatagagtacgagcca

SEQ ID NO: 8 - protein, 856 aa, Human immunodeficiency virus type 1
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 9 - protein, 498 aa, Lymphocytic choriomeningitis virus
MGQIVTMFEALPHIIDEVINIVIIVLIVITGIKAVYNFATCGIFALISFLLLAGRSCGMYGLKGPDIYKGVYQFKSVEFDMSHLNLTMPNACSANNSHHYISMGTSGLELTFTNDSIISHNFCNLTSAFNKKTFDHTLMSIVSSLHLSIRGNSNYKAVSCDFNNGITIQYNLTFSDAQSAQSQCRTFRGRVLDMFRTAFGGKYMRSGWGWTGSDGKTTWCSQTSYQYLIIQNRTWENHCTYAGPFGMSRILLSQEKTKFFTRRLAGTFTWTLSDSSGVENPGGYCLTKWMILAAELKCFGNTAVAKCNVNHDAEFCDMLRLIDYNKAALSKFKEDVESALHLFKTTVNSLISDQLLMRNHLRDLMGVPYCNYSKFWYLEHAKTGETSVPKCWLVTNGSYLNETHFSDQIEQEADNMITEMLRKDYIKRQGSTPLALMDLLMFSTSAYLVSIFLHLVKIPTHRHIKGGSCPKPHRLTNKGICSCGAFKVPGVKTVWKRR

SEQ ID NO: 10 - DNA, 180 bp, Lymphocytic choriomeningitis virus
cttttgatgttttcaacatcagcatatctaatcagcatctttctgcatcttgtgaagataccaacacatagacacataaagggcggttcatgtccaaagccacaccgcttgaccaacaaggggatctgtagttgtggtgcattcaaggtgcctggtgtaaaaactatctggaaaagacgc

SEQ ID NO: 11 - DNA, 31 bp, Artificial
  primer
aaggatccgccaccatgggcgttacaggaat

SEQ ID NO: 12 - DNA, 24 bp, Artificial
  primer
gtcacgacaaactagtttgtcgac

SEQ ID NO: 13 - DNA, 25 bp, Artificial
  primer
gggaattcttactttccaagtcggt

SEQ ID NO: 14 - DNA, 29 bp, Artificial
  primer
ggatggagaagctctattgcctctttttt

SEQ ID NO: 15 - DNA, 27 bp, Artificial
  primer
ggatggagaattgcctcttttttcttt

SEQ ID NO: 16 - DNA, 27 bp, Artificial
  primer
ggatggagatggaaaagctctattgcc

SEQ ID NO: 17 - DNA, 27 bp, Artificial
  primer
ggatggagatccaaaaatccaatcgag

SEQ ID NO: 18 - DNA, 27 bp, Artificial
  primer
ggatggagattttttggtgatactggg

SEQ ID NO: 19 - DNA, 27 bp, Artificial
  primer
tgtatatgccgagttggtatccatctt

SEQ ID NO: 20 - DNA, 27 bp, Artificial
  primer
tttgtctttcgagttggtatccatctt

SEQ ID NO: 21 - DNA, 27 bp, Artificial
  primer
ggatggagacttttgatgttttcaaca

SEQ ID NO: 22 - DNA, 28 bp, Artificial
  primer
gggaattctcagcgtcttttccagatag

SEQ ID NO: 23 - DNA, 28 bp, Artificial
  primer
tgtatatgccgattagtccaatttgtta

SEQ ID NO: 24 - DNA, 29 bp, Artificial
  primer
gggaattctatagagcctggaccactgat

SEQ ID NO: 25 - DNA, 29 bp, Artificial
  primer
gggaattctatggctcgtactctataggc

SEQ ID NO: 26 - DNA, 17 bp, Artificial
  primer
tgggaccggactgctgt

SEQ ID NO: 27 - DNA, 27 bp, Artificial
  primer
aatagagcttctccatcctgtccacca

SEQ ID NO: 28 - DNA, 27 bp, Artificial
  primer
agaggcaattctccatcctgtccacca

SEQ ID NO: 29 - DNA, 27 bp, Artificial
  primer
gcttttccatctccatcctgtccacca

SEQ ID NO: 30 - DNA, 27 bp, Artificial
  primer
atttttggatctccatcctgtccacca

SEQ ID NO: 31 - DNA, 27 bp, Artificial
  primer
accaaaaaatctccatcctgtccacca

SEQ ID NO: 32 - DNA, 28 bp, Artificial
  primer
accaactcggcatatacagaataaagcg

SEQ ID NO: 33 - DNA, 27 bp, Artificial
  primer
accaactcgaaagacaaatttgcatat

SEQ ID NO: 34 - DNA, 28 bp, Artificial
  primer
gactaatcggcatatacagaataaagcg

SEQ ID NO: 35 - DNA, 27 bp, Artificial
  primer
catcaaaagtctccatcctgtccacca

SEQ ID NO: 36 - DNA, 28 bp, Artificial
  primer
tgaattcctagcatatacagaataaagc

SEQ ID NO: 37 - DNA, 30 bp, Artificial
  primer
tttttcatgatttttttggtgatactgggc

SEQ ID NO: 38 - DNA, 31 bp, Artificial
  primer
tttttggccaacttttttggtgatactgggc

SEQ ID NO: 39 - DNA, 25 bp, Artificial
  primer
gggaattcttactttccaagtcggt

SEQ ID NO: 40 - DNA, 17 bp, Artificial
  primer
ggacccgtctagtggct

SEQ ID NO: 41 - DNA, 29 bp, Artificial
  primer
agttcttcttgttagatgcgacactgcag

SEQ ID NO: 42 - DNA, 29 bp, Artificial
  primer
aattgcttctgttagatgcgacactgcag

SEQ ID NO: 43 - DNA, 27 bp, Artificial
  primer
tgtgatcagtgtggcaagggttgttag

SEQ ID NO: 44 - DNA, 29 bp, Artificial
  primer
cgagttcttcttacaactgtgaaagacaa

SEQ ID NO: 45 - DNA, 27 bp, Artificial
  primer
agttcttctctccccgattgttgtatc

SEQ ID NO: 46 - DNA, 27 bp, Artificial
  primer
agttcttctggggttgaccttccaaat

SEQ ID NO: 47 - DNA, 27 bp, Artificial
  primer
agttcttctgctccttttcccacttgt

SEQ ID NO: 48 - DNA, 27 bp, Artificial
  primer
agttcttctctcattcagctggagcag

SEQ ID NO: 49 - DNA, 27 bp, Artificial
  primer
agttcttctggtcaaattgtcaacctc

SEQ ID NO: 50 - DNA, 27 bp, Artificial
  primer
agttcttcttccaaaaccggtagcctg

SEQ ID NO: 51 - DNA, 29 bp, Artificial
  primer
aatagagctttgtctccatcctgtccacc

SEQ ID NO: 52 - DNA, 27 bp, Artificial
  primer
catctaacaagaagaactcgaagagaa

SEQ ID NO: 53 - DNA, 28 bp, Artificial
  primer
catctaacagaagcaattgtcaatgctc

SEQ ID NO: 54 - DNA, 27 bp, Artificial
  primer
cttgccacactgatcacaggcgggaga

SEQ ID NO: 55 - DNA, 28 bp, Artificial
  primer
cacagttgtaagaagaactcgaagagaa

SEQ ID NO: 56 - DNA, 27 bp, Artificial
  primer
atcggggagagaagaactcgaagagaa

SEQ ID NO: 57 - DNA, 27 bp, Artificial
  primer
gtcaaccccagaagaactcgaagagaa

SEQ ID NO: 58 - DNA, 27 bp, Artificial
  primer
aaaaggagcagaagaactcgaagagaa

SEQ ID NO: 59 - DNA, 27 bp, Artificial
  primer
ctgaatgagagaagaactcgaagagaa

SEQ ID NO: 60 - DNA, 27 bp, Artificial
  primer
aatttgaccagaagaactcgaagagaa

SEQ ID NO: 61 - DNA, 27 bp, Artificial
  primer
ggttttggaagaagaactcgaagagaa

SEQ ID NO: 62 - DNA, 27 bp, Artificial
  primer
tttctcgagttttctcactaggagact

SEQ ID NO: 63 - DNA, 27 bp, Artificial
  primer
tttctcgagtaggagacttgcaggcac

SEQ ID NO: 64 - DNA, 17 bp, Artificial
  primer
gcaacagctgtattccc

SEQ ID NO: 65 - DNA, 25 bp, Artificial
  primer
aattgaaccatctcgagtagcaccc

SEQ ID NO: 66 - DNA, 33 bp, Artificial
  primer
cccactccattccggactcgggattccacctga

SEQ ID NO: 67 - protein, 11 aa, Artificial
  epitope
ZYQVNNLEEIC